(19) United States
(12) Patent Application Publication
(10) Pub. No.: US 2012/0328628 A1
(43) Pub. Date: Dec. 27, 2012

(54) ANTIBODIES TO CONFORMATIONALLY TRAPPED PROTEINS

(75) Inventors: James A. Wells, San Francisco, CA (US); Junjun Gao, Mountain View, CA (US)

(73) Assignee: The Regents of the University of California, Oakland, CA (US)

(21) Appl. No.: 12/307,906

(22) PCT Filed: Jul. 6, 2007
(86) PCT No.: PCT/US2007/072988

§ 371 Date: Oct. 30, 2009

Related U.S. Application Data
(60) Provisional application No. 60/819,139, filed on Jul. 7, 2006.

(51) Int. Cl. C07K 1/00; C07H 21/04; C07H 21/02; C07K 16/00; C07K 2/00; A61K 38/02; A61K 39/395; A61K 31/7105; A61K 31/711; C07K 1/14; A61K 39/00; A61P 25/16; A61P 35/00; A61P 25/00; A61P 9/10; A61P 29/00; A61P 31/00; C40B 30/04; G01N 33/566; C07H 1/00

(52) U.S. Cl. 424/172.1; 530/389.1; 530/350; 530/395; 536/23.1; 530/388.1; 530/387.2; 530/389.3; 514/1.1; 514/20.9; 514/44 R; 514/19.3; 514/17.7; 514/16.4; 514/15.4; 530/413; 424/184.1; 530/402; 506/9; 436/501; 435/7.92

(57) ABSTRACT

The present invention provides methods for generating antibodies to specific conformations of proteins. The conformation specific antibodies of the invention can be put to a variety of uses including diagnosis and treatment of diseases and for screening for compounds that induce conformational changes in proteins upon binding.

### CROSS-REFERENCES TO RELATED APPLICATIONS

The present application claims the benefit of U.S. Ser. No. 60/819,139, filed Jul. 7, 2006 herein incorporated by reference in its entirety.

### STATEMENT AS TO RIGHTS TO INVENTIONS MADE UNDER FEDERALLY SPONSORED RESEARCH AND DEVELOPMENT

Not Applicable.

### BACKGROUND OF THE INVENTION

Proteins are dynamic and can exist in multiple conformations. These conformational changes are often functionally important and reflect allosteric regulation that turns proteins on or off. Virtually every protein class undergoes some form of allosteric regulation. For example, the class of proteases known as caspases that control cell death and inflammation exist as inactive zymogens in the cell until they are activated by proteolysis. This causes a conformational transition that converts them to the active enzyme. The class of proteins known as kinases are generally in an inactive conformation in the cell until they are phosphorylated. This causes a conformational transition allowing them to become activated. As further examples, metabolic enzymes are highly regulated by reversible binding of small molecule effectors that turn them on or off.

The ability to manipulate allosteric transitions in proteins and to isolate particular conformational states of proteins would be very useful for both basic research as well as therapeutic applications. For example, if one had a pure form of a specific protein conformation it would be more amenable to x-ray crystallography, and thus, one could view the conformational transition directly.

Conformational states of proteins that are specific to particular disease states have been identified in a variety of pathologies. Prime examples of such diseases include those caused by prions. Prions are known to be responsible for a class of diseases known as transmissible spongiform encephalopathies (TSEs). Examples of TSEs include: scrapie (a disease of sheep), Creutfeldt-Jakob disease (CJD) in humans, and bovine spongiform encephalopathy (BSE or mad cow disease). The protein responsible for the disease is known as PrP, a protein which is also found in normal mammalian cells. Recent studies indicate that one mechanism for transmission of a prion disease is that upon infection, a diseased conformation of PrP (termed PrP^{Sc}) interacts with the normal PrP form found in the host (termed PrP^{C}), thereby converting the normal conformation of PrP^{C} into the diseased conformation present in PrP^{Sc}. Other prion diseases are inherited due to mutations in the host PrP gene, which render the encoded PrP protein more susceptible to the adoption of the diseased conformation. For a review of prion diseases, see “Prion diseases of humans and animals: their causes and molecular basis”. Annual Review of Neuroscience, 24: 519-50 (2001). The ability to detect and control the conformational states of PrP would be of clear benefit in the diagnosis and treatment of this class of diseases.

As another example, the caspases can become inappropriately and acutely activated during stroke, myocardial infarction or Parkinson's disease. Caspases are a class of cysteine proteases that cleave aspartate-containing substrates in a variety of physiological processes. Many of the caspases are held in an inactive form as a zymogen until they are activated by proteolytic cleavage, which converts the inactive caspase into an active conformation, allowing caspase cleavage of downstream targets. While inappropriate expression of particular caspases can lead to pathological states, the expression of others in an active form is necessary to induce programmed cell death in cancer cells. Thus, the ability to direct the conformational states of caspases into an inactive form would be beneficial to prevent tissue damage in some disease conditions such as those listed above, while the promotion of an active state conformation in cancer cells would be desirable.

As a further example, many receptors undergo a conformation change from an inactive into an active form upon binding of a ligand. G-protein coupled receptors (GPCRs) respond to a wide variety of extracellular signals. Upon ligand binding, these receptors undergo a conformational change, thereby relaying information to intracellular signal transduction pathways to effect an appropriate cellular response. GPCRs are involved in a wide range of physiological processes including cell growth, vision, smell, learning and memory, and inflammation. It would be of benefit to be able to maintain such receptors in an active or inactive conformation depending on physiological or pathophysiological conditions. For instance, it would be beneficial to be able to switch an actively signaling GPCR that mediates cell growth in a cancer cell into an inactive conformation.

From the foregoing discussion, it is clear that it would be desirable to have methods and reagents with which to identify, manipulate, and isolate the conformational state of proteins for diagnostic, treatment, and research purposes, among others. The invention disclosed herein addresses these and other needs.

### SUMMARY OF THE INVENTION

In one embodiment, a method of generating a protein binding domain that specifically binds to a protein in a specific conformational state is provided employing the steps of contacting a protein or a fragment thereof with a modifying agent that fixes the conformational state of the protein, and generating protein binding domains to the protein bound to the modifying agent, whereby the protein binding domains are specific for the conformational state of the protein. In different aspects, the protein binding domain can be an antibody, or fragment thereof, protein A, protein G, anykrin repeat domains, Fibronectin III domains, DNA, and RNA. In other aspects, the protein can be an inflammatory protein, a metabolic enzyme, a programmed cell death protein, a G-protein coupled receptor, an antibody, a blood coagulation factor, a cellular receptor, a coagulation factor, a protease, an extracellular protein or enzyme, a transcription factor, a cytoskeleton protein, a hormone receptor, a complement fixation protein, kinases and phosphatases. In other aspects, the programmed cell death protein can be caspase 1, 4, or 5. In additional aspects, the G-protein coupled receptor is a C5a receptor. In other aspects, the conformational state of the protein is active or inactive.

In another embodiment, a method of generating an antibody that specifically binds to a protein in a specific conformational state is provided employing the steps of contacting a protein or a fragment thereof with a modifying agent that fixes the conformational state of the protein, and generating antibodies to the protein bound to the modifying agent, whereby the antibodies are specific for the conformational state of the protein. In various aspects, the protein can be an inflammatory protein, a metabolic enzyme, a programmed cell death protein, a G-protein coupled receptor, an antibody, a blood coagulation factor, a cellular receptor, a coagulation factor, a protease, an extracellular protein or enzyme, a transcription factor, a cytoskeleton protein, a hormone receptor, a complement fixation protein, kinases and phosphatases. In other aspects, the programmed cell death protein can be caspase 1, 4, or 5. In additional aspects, the G-protein coupled receptor is a C5a receptor. In other aspects, the conformational state of the protein is active or inactive.

In some embodiments, the modifying agent is an agent that reacts with thiol, amino, or carboxyl groups on the protein. In further aspects, the binding of the modifying agent to the protein can be reversible or irreversible.

In other embodiments, a method of decreasing the activity of a protein by contacting the protein with the protein binding domain or antibody of the embodiments and aspects described above is provided. In some aspects, a method of increasing the activity of a protein by contacting the protein with the protein binding domain or antibody of the embodiments and aspects described above is provided.

In yet further embodiments, an antibody produced by the embodiments and aspects described above is provided. In various aspects, the antibody can be monoclonal or polyclonal.

In additional embodiments, a method for diagnosing a disease in a subject is provided by contacting a sample from the subject with the protein binding domain or antibody of the embodiments and aspects described above, where the protein binding domain or antibody binds to a form of the protein present in the disease and is indicative of presence of the disease in the subject. In various aspects, the disease can be cancer, autoimmune disease, Parkinson's disease, stroke, myocardial infarction, chronic inflammation, prion infection, neurological disease, renal disease, and infectious disease.

In other embodiments, a kit for diagnosing a disease using the protein binding domain or antibody of the embodiments and aspects described above is provided.

In yet other embodiments, a method of treating or preventing a disease by administering a therapeutically effective amount of the protein binding domain or antibody of the embodiments and aspects described above is provided. In various aspects, the disease can be cancer, autoimmune disease, Parkinson's disease, stroke, myocardial infarction, chronic inflammation, prion infection, neurological disease, renal disease, and infectious disease.

In still further embodiments, a method of purifying a protein in a specific conformational state by contacting a population of proteins with a plurality of conformational states with the protein binding domain or antibody of the embodiments and aspects described above, isolating the complex of the antibody bound to the protein, and eluting the protein from the antibody, where at least 50%, or preferably at least 60%, 70%, 80%, 90%, or 99%, of the resulting protein is in the specific conformational state.

In another embodiment, a method for screening for compounds that induce a specific conformational state of a protein by contacting a test compound with the protein, contacting the protein in the presence or absence of the test compound with the protein binding domain or antibody of the embodiments and aspects described above, and detecting the binding of the protein binding domain or antibody to the protein, where increased binding of the protein binding domain or antibody to the protein in the presence of the compound as compared to when the compound is absent indicates the adoption of the specific conformational state by the protein in the presence of the test compound.

### BRIEF DESCRIPTION OF THE DRAWINGS

FIG. 1 shows a schematic diagram that illustrates disulfide trapping.

FIG. 2 shows a comparison of the surface structure of caspases.

FIG. 3 shows a schematic diagram of the conformational states for caspase-7.

FIG. 4 shows a comparison of ligand-bound and ligand-free caspase-1 surfaces tructures and the location of a cysteine in the central cavity.

FIG. 5 shows a SDS-PAGE analysis of recombinant caspase-1, -4, and -5.

FIG. 6 shows the structure of Compound #34 bound to Cys331 in caspase-1. The compound projects from Cys331 across the dimer interface in a trans-configuration.

FIG. 7 shows the residues forming a H-bond network and salt bridge near the allosteric pocket of caspase-1. The active form of caspase-1 on the left contains many H-bond interactions that are not preserved in the allosteric structure (right). These residues are the subject of mutagenesis studies proposed to define the components of allosteric circuitry. A dashed line indicates a distance of less than 3.2 Å between two polar residues.

FIG. 8 shows the construction of half-labeled caspase-1.

FIG. 9 shows titration of caspase-1 with z-VAD-FMX.

FIG. 10 shows a schematic diagram of the generation of conformation specific antibodies by phage display.

FIG. 11 shows the heavy chain and light chain CDR sequences of Fabs. Only those positions that were randomized in the Fab libraries are shown.

FIG. 12 shows activation or inhibition of caspase-1 by conformationally specific Fabs. Caspase-1 at 5 nM concentration was pre-incubated with 2-fold dilutions of on-state Fab, off-state Fab or control Fab for 1 hr before adding the fluorogenic substrate (Ac-WEHD-AMC, 100 uM for on-state Fab assay and 10 uM for off-state Fab assay).

FIG. 13 shows the specificity of allosteric probes for caspase-1, -4 and -5. Panel A indicates the conservation of residues lining the allosteric pocket of the inflammatory caspases. The bridge composed if Ar286 and Glu390 is indicated with stars, the allosteric cysteine is boxed and the active site cysteine is indicated with an arrow. Panel B shows the percent overall sequence identity. Panel C indicates the specificity of compound #11 for capase-1, -4 and -5. Labeling of total free-cysteine was measured by mass spectrometry after treating the enzyme with increasing concentrations of compound #11. Some minor labeling artifact for capase-4 and caspase-5 at the highest concentrations are the result of mass spectral noise. Panel D indicates the percent activity remaining in enzyme treated with compound #11 at 100 uM for 1 hr, with labeling levels indicated by Panel C.

FIG. 14 shows the synthesis and activity of a cell permeable analog of compound #11. An analog of compound #11 (1M-11) with a neutral imidazole cap is shown in panel A. Panel B shows the effect of 1M-11 on IL-1β processing and panel C show the results of THP-1 cell challenge with 1M-11 in the presence of a transiently expressed wild-type or mutant form of pro-caspase-2. Transfected cells expressing either wild-type pro-caspase-1 or C331A were primed with LPS followed by treatment with increasing concentrations of IM-11. After a 45 min incubation with 1M-11, secretion of IL-1β was stipulated with ATP and secreted IL-1β was analyzed by western blotting. The amount of secreted IL-10 in the no compound control lane of C331A is lower that observed in the WT. This is due to a 5-fold decrease in kinetic activity of the mutant vs. the WT, as observed in kinetic analysis of the recombinant enzyme.

FIG. 15 shows the quantification of cellular uptake of IM-11 by THP-1 cells and reactivity of IM-11 with caspase-1 in the presence of a glutathione redox buffer. The results of quantification of four potential cellular derivatives of IM-11 in cell extracts after treatment with 25 micromolar IM-11 are shown.

FIG. 16 shows (A) A scatter plot of the percent inhibition of the 10,000 compounds screened against caspase-1 in the screen at the Small Molecule Discovery Center at UCSF. Points below the 50% inhibition line were selected for a follow-up secondary screen. Points at zero show positive controls with a known caspase-1 covalent inhibitor. (B) Hits from the primary screen were tested in the secondary screen for IC₅₀ values. The IC₅₀ plots for a selection of compounds from the primary screen are shown. A range of IC₅₀s is observed, with some compounds failing to show activity in the follow-up time-resolved assay. (C) Compounds are predicted to be either active site or allosteric site binders. Those showing no activity or other undesirable properties are discarded.

FIG. 17 shows the use of conformation-specific Fabs to probe the natural states of caspases-1. Fabₒₙ and Fab_{off} represent “on”-state and “off”-state Fab respectively.

FIG. 18 shows a model of dynamic states of caspase-1 upon substrate binding.

FIG. 19 shows the synthetic strategy for making analogues of compound #11.

FIG. 20 shows the strategy for making soluble compounds from a modified version of compound #11 using covalent extenders.

FIG. 21 shows the strategy for testing caspase specific probes in cell extracts or intact cells.

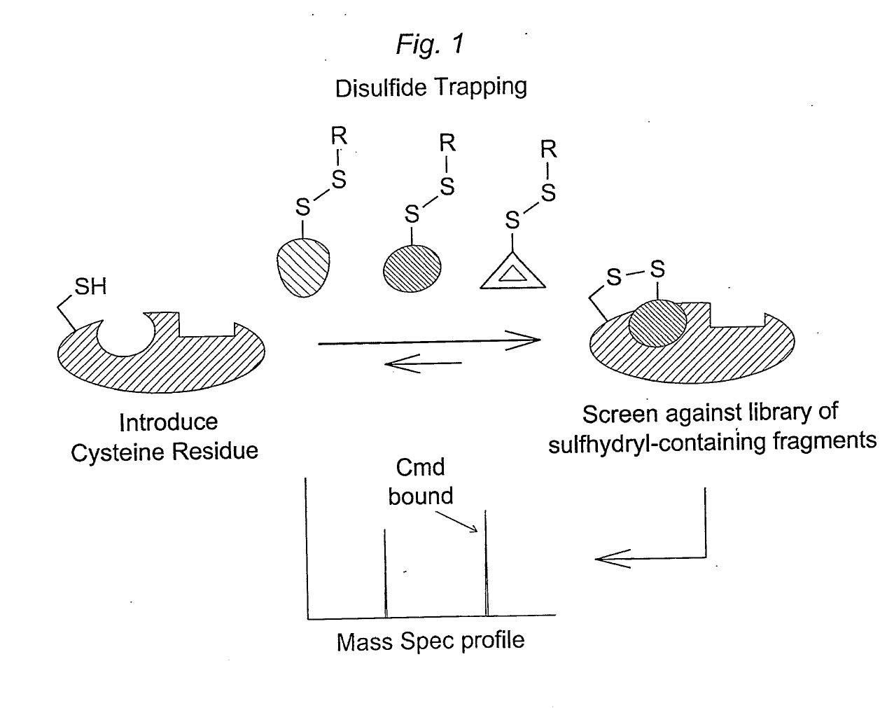

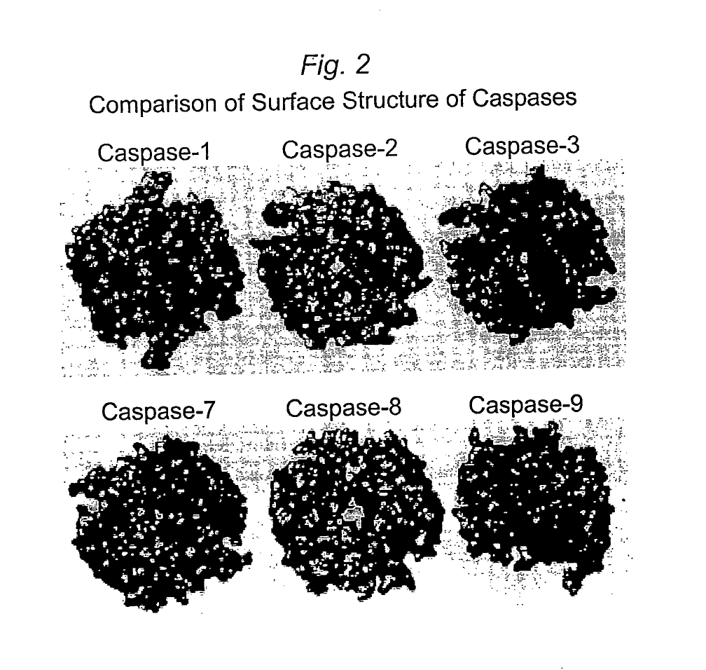

### DETAILED DESCRIPTION OF THE INVENTION

### I. Definitions

The term “conformation” or “conformational state” of a protein refers generally to the range of structures that a protein may adopt at any instant in time. On of skill in the art will recognize that determinants of conformation or conformational state include a protein's primary structure as reflected in a protein's amino acid sequence (including modified amino acids) and the environment surrounding the protein. The conformation or conformational state of a protein also relates to structural features such as protein secondary structures (e.g., α-helix, β-sheet, among others), tertiary structure (e.g., the three dimensional folding of a polypeptide chain), and quaternary structure (e.g., interactions of a polypeptide chain with other protein subunits). Post-translational and other modifications to a polypeptide chain such as ligand binding, phosphorylation, sulfation, or glycosylation, among others can influence the conformation of a protein. Furthermore, environmental factors, such as pH, salt concentration, ionic strength, and osmolarity of the surrounding solution, and interaction with other proteins and co-factors, among others, can affect protein conformation. The conformational state of a protein may be determined by either functional assay for activity or binding to another molecule or by means of physical methods such as X-ray crystallography, NMR, or spin labeling, among other methods. For a general discussion of protein conformation and conformational states, please refer to Cantor and Schimmel, Biophysical Chemistry, Part I: The Conformation of Biological. Macromolecules, W.H. Freeman and Company, 1980, and Creighton, Proteins: Structures and Molecular Properties, W.H. Freeman and Company, 1993.

A specific conformational state is any subset of the range of conformations or conformational states that a protein may adopt.

“Allostery” or “allosteric regulation” generally refers to the phenomenon in which a binding event at one site of a protein propagates a conformational change to a second site in a protein. Examples of allostery are found in a wide range of proteins including bacterial repressor proteins, hemoglobin, many metabolic enzymes, signaling enzymes, molecular motors, G-protein couples receptors (GPCRs), and hormone receptors, among others.

A “modifying agent” is a compound that interacts with a protein to fix or trap the protein in a specific conformational state. The interaction maybe covalent or non-covalent. A modifying agent may be one that reacts with thiol, amino, or carboxyl groups, or other functionalities on a protein. A modifying agent can also be affinity labeling reagents, such as photoaffinity labeling agents. Examples of non-covalent modifying agents include natural or non-natural exogenous binding ligands, e.g., small organic molecues which can bind to the protein to lock the protein in a specific conformation. Such non-covalent modifying agents may be contacted with a protein at saturating concentrations.

A protein that is “conformationally trapped” or “fixed” is one that is held in a subset of the possible conformations that it could otherwise assume, generally due to the effects of the interaction of the protein with a modifying agent.

An antibody that binds to a specific conformation or conformational state of a protein refers to an antibody that binds with a higher affinity to a protein in a subset of conformations or conformational states than to other conformations or conformational states that a protein may assume.

The conformational state of a protein is “active” when a subset of conformational states increases, opens, activates, facilitates, enhances activation, enhances binding, or up regulates the protein's activity by at least 10% over another conformation state of the protein.

The conformational state of a protein is “inactive” when a subset of conformational states decreases, closes, deactivates, hinders, diminishes activation, or diminishes binding, or down regulates the protein's activity by at least 10% over another conformation state of the protein.

The term “protein binding domain” refers generally to any molecule that is able to bind specifically to a protein or peptide. A variety of molecules can function as protein binding domains, including, but not limited to, proteins, peptides, nucleic acids, and sugars. The terms “molecular scaffold” or “protein scaffold” refer generally to folding units that form structures, particularly protein or peptide structures, that comprise frameworks for the binding of another molecule, for instance a protein. (See, e.g., Skerra, J. Molecular Recognition, 13:167-187 (2000), for review.)

Examples of protein binding domains which are known in the art include, but are not limited to: antibodies, and fragments thereof, protein A, protein G, ankyrin repeats, fibronectin type III repeats, model peptides and proteins, DNA, and RNA. Other examples include: members of the immunoglobulin superfamily, protease inhibitors, helix-bundle proteins, disulfide-knotted peptides, and lipocalins. (See, e.g., Skerra, J. Molecular Recognition, 13:167-187 (2000); Starovasnik et al., Proc. Natl. Acad. Sci. USA, 94: 10080-10085 (1997); Binz et al., Nature Biotech., 22: 575-582 (2004); Koide et al., J. Mol. Biol., 284: 1141-1151 (1998)). Frequently, when generating a particular type of protein binding domain using selection methods, combinatorial libraries comprising a consensus or framework sequence containing randomized potential interaction residues are used to screen for binding to a molecule of interest, such as a protein.

The term “antibody” refers to a polypeptide encoded by an immunoglobulin gene, or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Examples of antibody functional fragments include, but are not limited to, complete antibody molecules, antibody fragments, such as Fv, single chain Fv (scFv), complementarity determining regions (CDRs), V_{L} (light chain variable region), V_{H} (heavy chain variable region), Fab, F(ab)₂′ and any combination of those or any other functional portion of an immunoglobulin peptide capable of binding to target antigen (see, e.g., Fundamental Immunology (Paul ed., 3d ed. 1993). As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)). The term antibody also includes bivalent or bispecific molecules, diabodies, triabodies, and tetrabodies. Bivalent and bispecific molecules are described in, e.g., Kostelny et al. (1992) J Immunol 148:1547, Pack and Pluckthun (1992) Biochemistry 31:1579, Hollinger et al., 1993, supra, Gruber et al. (1994) J Immunol:5368, Zhu et al. (1997) Protein Sci 6:781, Hu et al. (1996) Cancer Res. 56:3055, Adams et al. (1993) Cancer Res. 53:4026, and McCartney, et al. (1995) Protein Eng. 8:301.

References to “V_{H}” or a “VH” refer to the variable region of an immunoglobulin heavy chain, including an Fv, scFv, a disulfilde-stabilized F_{V} (dsFv) or Fab. References to “V_{L}” or a “VL” refer to the variable region of an immunoglobulin light chain, including of an Fv, scFv, dsFv or Fab.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. The numbering of the light and heavy chain variable regions described herein is in accordance with Kabat (see, e.g., Johnson et al., (2001) “Kabat Database and its applications: future directions” Nucleic Acids Research, 29: 205-206; and the Kabat Database of Sequences of Proteins of Immunological Interest, Feb. 22, 2002 Dataset).

The positions of the CDRs and framework regions are determined using various well known definitions in the art, e.g., Kabat, Chothia, international ImMunoGeneTics database (IMGT), and AbM (see, e.g., Johnson et al., supra; Chothia and Lesk, J. Mol. Biol. 196:901-917 (1987); Chothia et al., Nature 342, 877-883 (1989); Chothia et al., J. Mol. Biol. 227, 799-817; Al-Lazikani et al., J. Mol. Biol 1997, 273(4)). Definitions of antigen combining sites are also described in the following: Ruiz et al., IMGT, the international ImMunoGeneTics database. Nucleic Acids Res., 28: 219-221 (2000); and Lefranc, M.-P. IMGT, the international ImMunoGeneTics database. Nucleic Acids Res. 29(1):207-9 (2001); MacCallum et al., J. Mol. Biol., 262 (5):732-745 (1996); and Martin et al, PNAS USA 86:9268-9272 (1989); Martin, et al, Methods Enzymol., 203:121-153, (1991); Pedersen et al, Immunomethods, 1, 126, (1992); and Rees et al, In Sternberg M. J. E. (ed.), Protein Structure Prediction. Oxford University Press, Oxford, 141-172 1996).

A “chimeric antibody” as used herein, refers to an antibody whose light and heavy chain genes have been constructed, typically by genetic engineering, from immunoglobulin variable and constant region genes belonging to different species. For example, the variable segments of the genes from a mouse monoclonal antibody may be joined to human constant segments, such as gamma 1 and gamma 3. A therapeutic chimeric antibody thus comprises a hybrid protein that may be composed of, for example, the variable or antigen-binding domain from a mouse antibody and the constant or effector domain from a human antibody. Naturally, this example is not limiting. Combinations of variable and constant domains may involve mammalian species other than mouse and human as well.

The term “humanized antibody” refers to an immunoglobulin molecule comprising a human-like framework region and one or more CDR's from a non-human (usually a mouse or rat) immunoglobulin. Constant regions need not be present, but if they are, they must be substantially identical to human immunoglobulin constant regions, i.e., at least about 85-90%, preferably about 95% or more identical. The resultant humanized antibody is expected to bind to the same antigen as the donor antibody that provides the CDR's. Thus, used herein, the term “humanized antibody” is an embodiment of chimeric antibodies wherein substantially less than an intact human variable domain has been substituted by the corresponding sequence from a non-human species. In practice, humanized antibodies are typically human antibodies in which some CDR residues are substituted by residues from analogous sites in rodent antibodies.

The term “hybridoma cell line” refers to a permanent cell line derived from the fusion of a cultured a neoplastic lymphocyte (e.g. a mouse plasmacytoma cell) and specific antibody producing cell i.e. a primed B or T lymphocyte. All of the cells of a particular hybridoma cell line express the specific immune potential of the B or T lymphocyte. For example, a B cell hybridoma continuously secretes pure monoclonal antibody of a specificity determined by the immune potential of the parental B cell. Thus, such a cell line may be used for the large scale production of the specific antibodies produced by the B cell. Hybridoma cell lines are permanently adapted to growth in culture, but may also form specific antibody producing tumors in vivo.

The term “effector moiety” means the portion of an immunoconjugate intended to have an effect on a cell targeted by the targeting moiety or to identify the presence of the immunoconjugate. Thus, the effector moiety can be, for example, a therapeutic moiety, such as a cytotoxic agent or drug, or a detectable moiety, such as a fluorescent label.

The term “immunoconjugate” refers to a composition comprising an antibody linked to a second molecule such as a detectable label or effector molecule. Often, the antibody is linked to the second molecule by covalent linkage.

In the context of an immunoconjugate, a “detectable label” or “detectable moiety” refers to, a portion of the immunoconjugate which has a property rendering its presence detectable. For example, the immunoconjugate may be labeled with a radioactive isotope which permits cells in which the immunoconjugate is present to be detected in immunohistochemical assays. A “detectable label” or a “detectable moiety” is a composition detectable by spectroscopic, photochemical, biochemical, immunochemical, chemical, or other physical means. For example, useful labels include radioisotopes (e.g., ³H, ³⁵S, ³²P, ⁵¹Cr, or ¹²⁵I), fluorescent dyes, electron-dense reagents, enzymes (e.g., alkaline phosphatase, horseradish peroxidase, or others commonly used in an ELISA), biotin, digoxigenin, or haptens and proteins which can be made detectable, e.g., by incorporating a radiolabel into the peptide or used to detect antibodies specifically reactive with the peptide. An introduction to labels, labeling procedures, and detection of labels is found in Polak and Van Noorden Introduction to Immunocytochemistry, 2nd ed., Springer Verlag, NY (1997); and in Haugland Handbook of Fluorescent Probes and Research Chemicals, a combined handbook and catalogue Published by Molecular Probes, Inc. (1996).

The term “immunologically reactive conditions” includes reference to conditions which allow an antibody generated to a particular epitope to bind to that epitope to a detectably greater degree than, and/or to the substantial exclusion of, binding to substantially all other epitopes. Immunologically reactive conditions are dependent upon the format of the antibody binding reaction and typically are those utilized in immunoassay protocols or those conditions encountered in vivo (see Harlow & Lane, ANTIBODIES, A LABORATORY MANUAL, Cold Spring Harbor Press, New York (1988) and Harlow & Lane, USING ANTIBODIES, A LABORATORY MANUAL, Cold Spring Harbor Press, New York (1999), for a description of immunoassay formats and conditions that can be used to determine specific immunoreactivity). In some cases, the immunologically reactive conditions employed in the methods of the present invention may be “physiological conditions” which include reference to conditions (e.g., temperature, osmolarity, pH) that are typical inside a living mammal or a mammalian cell. While it is recognized that some organs are subject to extreme conditions, the intra-organismal and intracellular environment normally lies around pH 7 (i.e., from pH 6.0 to pH 8.0, more typically pH 6.5 to 7.5), contains water as the predominant solvent, and exists at a temperature above 0° C. and below 50° C. Osmolarity is within the range that is supportive of cell viability and proliferation.

The term “binding specificity,” “specifically binds to an antibody” or “specifically immunoreactive with,” when referring to an epitope, refers to a binding reaction which is determinative of the presence of the epitope in a heterogeneous population of proteins and other biologics. Thus, under designated immunoassay conditions, the specified antibodies bind to a particular epitope at least two times the background and more typically more than 10 to 100 times background. A variety of immunoassay formats may be used to select antibodies specifically immunoreactive with a particular protein or carbohydrate. For example, solid-phase ELISA immunoassays are routinely used to select antibodies specifically immunoreactive with a protein or carbohydrate (see, e.g. Harlow & Lane, supra).

It is understood that antibodies that bind to specific conformational states of proteins may be antibodies that have conservative amino acid substitutions relative to each other. Such “conservatively modified variants” are in addition to and do not exclude polymorphic variants, interspecies homologues, and alleles of the invention.

Conservative substitution tables providing functionally similar amino acids are well known in the art. For example, substitutions may be made wherein an aliphatic amino acid (G, A, I, L, or V) is substituted with another member of the group. Similarly, an aliphatic polar-uncharged group such as C, S, T, M, N, or Q, may be substituted with another member of the group; and basic residues, e.g., K, R, or H, may be substituted for one another. In some embodiments, an amino acid with an acidic side chain, E or D, may be substituted with its uncharged counterpart, Q or N, respectively; or vice versa. Each of the following eight groups contains other exemplary amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M)

(see, e.g., Creighton, Proteins (1984)).

The expression “conservatively modified variants” and it equivalents applies to both nucleic acid and amino acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

With respect to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid.

The terms “identical” or percent “identity,” in the context of two or more nucleic acid or polypeptide sequences, refer to two or more sequences or subsequences that are the same or have a specified percentage of amino acid residues or nucleotides that are the same (i.e., 60% identity, preferably 65%, 70%, 75%, 80%, 85%, 90%, 95%, 96%, 97%, 98%, or 99% identity over a specified region), when compared and aligned for maximum correspondence over a designated region as measured using one of the following sequence comparison algorithms or by manual alignment and visual inspection. Such sequences are then said to be “substantially identical.” This definition also refers to the compliment of a test sequence. Preferably, the identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

While any method known in the art for making such determinations may be used, for the purpose of the present invention, the BLAST algorithm, described in Altschul et al., J. Mol. Biol. 215:403-410 (1990) and Karlin et al., PNAS USA 90:5873-5787 (1993), and incorporated herein by reference, may be used preferentially for determining sequence identity according to the methods of the invention. A particularly useful BLAST program is the WU-BLAST-2 program (Altschul et al., Methods in Enzymology 266: 460-480 (1996) also incorporated herein by reference). WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity. A percent sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

A polypeptide is also considered to be substantially identical to a second polypeptide, for example, where the two peptides differ only by conservative substitutions. An indication that two nucleic acid sequences are substantially identical is that the two molecules or their complements hybridize to each other under stringent conditions, as described below. Another indication that two nucleic acid sequences are substantially identical is that the same primers can be used to amplify the sequence.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The term “test compound” or “drug candidate” or “modulator” or grammatical equivalents as used herein describes any molecule, either naturally occurring or synthetic, e.g., protein, oligopeptide (e.g., from about 5 to about 25 amino acids in length, preferably from about 10 to 20 or 12 to 18 amino acids in length, preferably 12, 15, or 18 amino acids in length), small organic molecule, siRNA, polysaccharide, lipid, fatty acid, polynucleotide, oligonucleotide, etc., to be tested in a drug assay. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

A “small organic molecule” refers to an organic molecule, either naturally occurring or synthetic, that has a molecular weight of more than about 50 daltons and less than about 2500 daltons, preferably less than about 2000 daltons, preferably between about 100 to about 1000 daltons, more preferably between about 200 to about 500 daltons.

### II. Introduction

In various embodiments, the present invention provides protein conformational state specific protein binding domains or antibodies. These protein binding domains or antibodies can be used to identify, manipulate, and isolate proteins in specific conformations. Accordingly, for instance, a conformation specific protein binding domain or antibody can be used as a tool to diagnose the presence of a protein with a conformation characteristic of a given disease in a patient sample. Conformation specific protein binding domains or antibodies can be used to purify large quantities of proteins in a particular conformation for studies such as X-crystallography. Conformation specific protein binding domains or antibodies may also be used in large screens for compounds that induce a protein to adopt a particular conformation. Such compounds would have a therapeutic benefit, for instance, if the compound is able to switch a protein from a conformation that causes a disease state into the normal form.

Conformation specific protein binding domains or antibodies may also be used therapeutically, for example, as vaccines or as pharmaceuticals that are able to revert aberrant conformations of proteins in various disease states back to their normal conformations. A number of protein therapeutics are known to effect the conformation of a target receptor or else are required to be in a particular conformation to be effective. Examples of such protein therapeutics include: Monoclonal Antibodies (e.g. Herceptin, Rituximab, Avastin, etc.), Erythropoietin, Insulin, Cytokines, Interleukins, Keratinocyte Growth Factor, Granulocyte-Colony Stimulating Factor, Growth Hormones, Somatotropin, Somatomedins (IGF), Blood Factors, Recombinant BMP, Luteinizing Hormone, Follicle-Stimulating Hormone, Human Chorionic Gonadotropin, Progestrone, Estrogen, Tissue Plasminogen Activators, and vaccines. These protein therapeutics are used to treat or prevent a variety of conditions such as cancer, inflammation, autoimmune diseases, bone repair, growth, reproductive system dysfunction, and viral infections, among others. Conformation specific protein binding domains or antibodies or compounds that induce particular protein conformations that may be identified using this invention may be used to treat diseases currently being treated by these protein therapeutics.

Alternatively, the protein binding domains or antibodies of this invention may be used to isolate conformational active forms of the pre-existing therapeutic agents described above. For example, the protein binding domains or antibodies of this invention can be used to isolate active conformations of proteins such as blood coagulation factors, receptors or enzymes, among others, thus eliminating the need for ligands, co-factors, or interaction with other protein subunits. As another use, the protein binding domains or antibodies of the invention may be used directly as vaccines.

In the practice of embodiments of this invention, one of skill in the art must first acquire sufficient amounts of conformationally trapped proteins to generate antibodies. A number of standard molecular biological, cell biological, and biochemical methods are known to the skilled artisan and may be used for this purpose. A protein of interest may be present in a variety of cells and tissues, either naturally, or by means of recombinant expression. The protein of interest is reacted with modifying agents that fix the protein a specific conformational state. The treatment of the protein with the modifying agent can occur on purified or partially purified preparations of the protein, or within a cell or tissue. The presence of a protein trapped in a particular conformation may be ascertained by a variety of biochemical and physical methods, such as X-ray crystallography, NMR and spin-labeling, among other methods. The conformationally trapped protein, with or without further purification, is used to generate antibodies. Among the methods available to generate antibodies are immunization of a suitable animal to generate monoclonal or polyclonal antibodies that recognize specific conformations of the protein. Other methods include phage display methodology, which may be used to isolate Fabs that recognize specific protein conformations. On of skill in the art will recognize that included within the scope of this invention are antibodies raised against the conformation specific antibodies described above, e.g., anti-idiotype antibodies.

Described below is a non-limiting set of standard methodologies available to the skilled artisan that may be used to practice this invention. Other non-limiting methods may be found in the Examples section.

### III. Production and Purification of Conformationally Trapped Proteins

A. Recombinant Expression of Proteins

To practice the methods of the invention, one of skill in the art must acquire sufficient amounts of conformationally trapped proteins for the purpose of preparing protein binding domains that bind to them or antibodies. One such approach is to produce large amounts of a protein using recombinant methods.

1. Generation of cDNAs Encoding Proteins or Fragments

One of skill in the art will recognize that given the vast amount of nucleic acid sequence information available from the human genome as well as from other species that the DNA encoding virtually any protein can be obtained from conventional methods such as library screening or PCR. The methods of molecular biology can be further utilized to generate either full length proteins or any desired fragments, including proteins and fragments with amino acid substitutions that may be favorable, such as the inclusion of cysteine residues for reaction with particular classes of modifying agents One of skill in the art will recognize that PCR and mutagenesis techniques can be used to manipulate a DNA sequence to add convenient restriction sites or to mutagenize a DNA sequence as desired. Detailed descriptions of standard molecular biological methods including PCR and mutagenesis techniques can be found, for example at Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)). In addition, kits for many molecular biological methods are commercially available.

2. Expression of Cloned Genes

To obtain high level expression of a cloned gene, one typically subclones the DNA sequence into an expression vector that contains a strong promoter to direct transcription, a transcription/translation terminator, and if for a nucleic acid encoding a protein, a ribosome binding site for translational initiation. Suitable bacterial promoters are well known in the art and described, e.g., in Sambrook et al., and Ausubel et al., supra. Bacterial expression systems are available in, e.g., E. coli, Bacillus sp., and Salmonella (Palva et al., Gene 22:229-235 (1983); Mosbach et al., Nature 302:543-545 (1983)). Kits for such expression systems are commercially available. Eukaryotic expression systems for mammalian cells, yeast, and insect cells are well known in the art and are also commercially available.

Selection of the promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of the protein encoding nucleic acid in host cells. A typical expression cassette thus contains a promoter operably linked to the nucleic acid sequence encoding a monomeric subunit and signals required for efficient polyadenylation of the transcript, ribosome binding sites, and translation termination. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as MBP, GST, and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Ban virus. Other exemplary eukaryotic vectors include pMSG, pAV009/A⁺, pMTO10/A⁺, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Expression of proteins from eukaryotic vectors can be also be regulated using inducible promoters. With inducible promoters, expression levels are tied to the concentration of inducing agents, such as tetracycline or ecdysone, by the incorporation of response elements for these agents into the promoter. Generally, high level expression is obtained from inducible promoters only in the presence of the inducing agent; basal expression levels are minimal. Inducible expression vectors are often chosen if expression of the protein of interest is detrimental to eukaryotic cells.

Some expression systems have markers that provide gene amplification such as thymidine kinase and dihydrofolate reductase. Alternatively, high yield expression systems not involving gene amplification are also suitable, such as using a baculovirus vector in insect cells, with a monomeric subunit encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells, if necessary.

Standard transfection methods are used to produce bacterial, mammalian, yeast or insect cell lines that express large quantities of protein, which can be purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983).

Any of the well-known procedures for introducing foreign nucleotide sequences into host cells may be used. These include the use of calcium phosphate transfection, polybrene, protoplast fusion, electroporation, biolistics, liposomes, microinjection, plasma vectors, viral vectors and any of the other well known methods for introducing cloned genomic DNA, cDNA, synthetic DNA or other foreign genetic material into a host cell (see, e.g., Sambrook et al., supra).

After the expression vector is introduced into the cells, the transfected cells are cultured under conditions favoring expression of the protein, which may be recovered from the culture using standard techniques identified below.

B. Purification of Expressed Proteins.

Proteins and/or fragments thereof can be purified from any suitable expression system or from a source that naturally expresses a protein of interest as described below. If desired, the protein may be purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook et al., supra). However, in the practice of this invention, purified or partially purified proteins are not required for either treatment with a modifying agent or generation of antibodies. If purification of the a conformationally trapped protein is desired, the presence of the modifying agent may be used to aid in the purification by allowing a skilled artisan to follow the location of the conformationally trapped protein during various purification steps such as those described below.

1. Purification of Proteins from Recombinant Bacteria

Recombinant proteins can be expressed by transformed bacteria in large amounts, typically after promoter induction; but expression can be constitutive. Promoter induction with IPTG is one example of an inducible promoter system. Bacteria are grown according to standard procedures in the art. Fresh or frozen bacteria cells are used for isolation of protein.

Proteins expressed in bacteria may form insoluble aggregates (“inclusion bodies”). Several protocols are suitable for purification of the expressed proteins from inclusion bodies. For example, purification of inclusion bodies typically involves the extraction, separation and/or purification of inclusion bodies by disruption of bacterial cells. The cell suspension can be lysed using 2-3 passages through a French Press; homogenized using a Polytron (Brinkman Instruments); disrupted enzymatically, e.g., by using lysozyme; or sonicated on ice. Alternate methods of lysing bacteria are apparent to those of skill in the art (see, e.g., Sambrook et al., supra; Ausubel et al., supra).

If necessary, the inclusion bodies are solubilized, and the lysed cell suspension is typically centrifuged to remove unwanted insoluble matter. Proteins that formed the inclusion bodies may be renatured by dilution or dialysis with a compatible buffer. Suitable solvents include, but are not limited to urea (from about 4 M to about 8 M), formamide (at least about 80%, volume/volume basis), and guanidine hydrochloride (from about 4 M to about 8 M). Some solvents which are capable of solubilizing aggregate-forming proteins, for example SDS (sodium dodecyl sulfate), 70% formic acid, are inappropriate for use in this procedure due to the possibility of irreversible denaturation of the proteins, accompanied by a lack of immunogenicity and/or activity.

Although guanidine hydrochloride and similar agents are denaturants, this denaturation is not irreversible and renaturation may occur upon removal (by dialysis, for example) or dilution of the denaturant, allowing re-formation of immunologically and/or biologically active protein. Other suitable buffers are known to those skilled in the art. One of skill in the art will recognize that optimal conditions for renaturation must be chosen for each protein. For example, if a protein is soluble only at low pH, renaturation can be done at low pH. Renaturation conditions can thus be adjusted for proteins with different solubility characteristics i.e., proteins that are soluble at neutral pH can be renatured at neutral pH. The expressed protein is separated from other bacterial proteins by standard separation techniques.

2. Standard Protein Separation Techniques for Purifying Proteins

a) Solubility Fractionation

Often as an initial step, particularly if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

b) Size Differential Filtration

The molecular weight of a given protein can be used to isolate it from proteins of greater and lesser size using ultrafiltration through membranes of different pore size (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

c) Column Chromatography

A protein can also be separated from other proteins on the basis of its size, net surface charge, hydrophobicity, and affinity for ligands. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

### IV. Trapping of Conformational States in Proteins

A modifying agent can be contacted with a protein of interest to fix the protein in a specific conformational state. The interaction of the modifying agent may be covalent or non-covalent and may be reversible or essentially irreversible. Functional groups on proteins that may serve as sites for attachment of modifying agents include sulfhydryl, amino, and carboxyl groups found on the side chains of various amino acids. Furthermore, other features of a protein such as charge, hydrophobicity, or hydrogen bonding potential, among others, may serve as a point of association between a modifying agent and a protein. As described in the Examples, in one embodiment of this invention, modifying agents that react with cysteine residues may be advantageous agents to use in the practice of this invention. Further examples of trapping of conformational states using modifying agents such as those which react with sulfhydryl groups may be found in: Erlanson et al., Annual Review of Biophys. Biomol. Struct., 33:199-223 (2004), Erlanson, et al., Curr. Opin. Chem. Biol., 8: 399-406 (2004), Erlanson et al., J. Med. Chem., 47: 3463-3482 (2004), Hardy et al., Proc. Nat'l. Acd. Sci., 34:12461-12466 (2004); Buck and Wells, Proc. Nat'l. Acd. Sci., 102: 2719-2724 (2005); Scheer et al., Proc. Nat'l. Acd. Sci., 103: 7595-7600 (2006), which are incorporated by reference in their entirety.

Alternatively, mutations can be introduced into a protein sequence to fix or trap the protein in a specific conformational state. For instance, glycine “hinge” points in proteins that undergo conformational transitions can be identified. By converting the flexible glycine residue to a less flexible alanine residue, the enzyme can be a specific conformation depending on the whether the dihedral angles are allowed in the Ramachandran diagram. Thus, one can create such locks on protein conformation by the identification of glycine residues that change conformation between two structures and introducing alanine substitutions that are differentially allowed.

Other types of mutations that can be introduced into a protein sequence to fix or trap the protein in a specific conformational state include the use of site directed mutagenesis to introduce residues that lock or stabilize a specific conformational state of a protein. Non-limiting examples of such mutations include: mutagenesis to stabilize subtilisin in a transition state (Braxton et al., J. Biol. Chem., 266: 11797-11800 (1991)); mutagenesis to stabilize the activated state of alcohol dehydrogenase (Ramaswamy, et al., Biochemistry, 38: 13951-13959 (1999)); mutagenesis to introduce stabilizing disulfide bonds into staphylococcal nuclease Hinck et al., Biochemistry, 35: 10328-10338 (1996)).

A variety of methods may be used to identify modifying agents or mutations that fix a protein in a specific conformation. Typically, an assay that provides a readily measured parameter is adapted to be performed in the wells of multi-well plates in order to facilitate the screening of members of a library of test compounds as described herein. Thus, in one embodiment, an appropriate number of cells can be plated or an appropriate amount of a purified protein is deposited into the cells of a multi-well plate, and the effect of a test compound on a detectable parameter reflecting protein conformation can be determined. Thus, for instance, if one conformation of a protein is active and another inactive, one may screen for wells where the addition of a test compound has affected the activity of the protein. Alternatively, methods such as resonance energy transfer can be used to identify conformational changes in the presence of a test compound that is reflected by a change in the distance between the donor and acceptor fluorophore. Other methods for detecting conformational trapping by a modifying agent include NMR and spin labeling measurements.

### V. Preparation of Antibodies to Conformationally Trapped Proteins

Methods of producing polyclonal and monoclonal antibodies that react specifically with proteins are known to those of skill in the art and can be readily adapted to generate conformation specific antibodies by using the conformationally trapped proteins described above as antigens (see, e.g., Coligan, Current Protocols in Immunology (1991); Harlow & Lane, supra; Goding, Monoclonal Antibodies: Principles and Practice (2d ed. 1986); and Kohler & Milstein, Nature 256:495-497 (1975). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)).

Methods of production of polyclonal antibodies are known to those of skill in the art. An inbred strain of mice (e.g., BALB/C mice) or rabbits is immunized with the protein (i.e., immunogen) using a standard adjuvant, such as Freund's adjuvant, and a standard immunization protocol. The animal's immune response to the immunogen preparation is monitored by taking test bleeds and determining the titer of reactivity to the protein. When appropriately high titers of antibody to the immunogen are obtained, blood is collected from the animal and antisera are prepared. Further fractionation of the antisera to enrich for antibodies reactive to the protein can be done if desired (see, Harlow & Lane, supra).

Monoclonal antibodies may be obtained by various techniques familiar to those skilled in the art. Briefly, spleen cells from an animal immunized with a desired antigen are immortalized, commonly by fusion with a myeloma cell (see, Kohler & Milstein, Eur. J. Immunol. 6:511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse, et al., Science 246:1275-1281 (1989).

Monoclonal antibodies and polyclonal sera are collected and titered against the immunogen protein in an immunoassay, for example, a solid phase immunoassay with the immunogen immobilized on a solid support. Typically, polyclonal antisera with a titer of 10⁴ or greater are selected and tested for their cross reactivity against non-conformationally trapped proteins or proteins trapped in a conformation different from that used to raise the antibody. Specific polyclonal antisera and monoclonal antibodies will usually bind with a K_{d} of at least about 0.1 mM, more usually at least about 1 μM, preferably at least about 0.1 μM or better, and most preferably, 0.01 μM or better.

Once antibodies specific to a particular conformational state are available, the antibodies can be sequenced, or can be manipulated so as create chimeric or humanized antibodies as described below.

### VI. Characterization of Monoclonal Antibodies

### A. Isotype Determination

Mammalian immunoglobins have been classified into five primary classes (IgG, IgM, IgA, IgD and IgE) according to differences in their heavy chain polypeptides. Several of these classes can be further divided into subclasses, e.g., IgG₁, IgG₂, IgG₃, IgG₄, IgA₁, and IgA₂. These classes can be identified based on their reaction to antisera. Similarly, mammalian light chain constant regions can be assigned to one of two clearly distinct isotypes based on their amino acid sequence and reactions to different antisera. These isotypes are called κ (kappa) and λ (lambda).

Because the biological functions and biochemical characteristics of classes and isotypes differ, distinguishing the classes and isotypes of an immunoglobulin molecule is critical. Although any immunoaffinity method known in the art for can be used to determine antibody isotypes. The following provides an example of isotyping using an ELISA to determine the isotype of mouse antibodies.

For the ELISA assay, anti-mouse immunoglobulin antibodies are coated onto each well of a 96-well microtiter plate that serves as a solid support. Sample mouse immunoglobulins in solution are added and captured by the anti-mouse antibodies. Specific anti-mouse isotyping antibodies are then introduced and allowed to bind to the mouse-anti-mouse antibody complex. Finally, an enzyme-tagged antibody that reacts specifically with the anti-isotyping antibodies is added, which, together with a colorimetric substrate, indicate the immunoglobulin isotype of the sample. Antibody isotyping is well known in the art and kits are commercially available (e.g., isotyping kits such as the Isodetect kit are available from Stratagene, La Jolla, Calif.).

B. Epitope Mapping

Regions of a given polypeptide that include an epitope can be identified using any number of epitope mapping techniques known in the art (see e.g., Epitope Mapping Protocols in Methods in Molecular Biology, Vol. 66 (Glenn E. Morris, ed., 1996) Humana Press, Totowa, N.J.) Methods for epitope mapping may include solving the crystal structure of an antibody-antigen complex or may involve analysis of vast libraries of random peptide sequences. However, the most convenient methods typically involve synthetic peptide based assays and competition assays.

Linear epitopes may be determined by synthesizing large numbers of peptides corresponding to portions of a protein molecule such as Vpr, on solid supports, and then reacting the peptides with antibodies while the peptides are attached to the supports. In this method, a set of overlapping peptides is synthesized, each corresponding to a small linear sequence of the antigen and arrayed on a solid phase. The panel of solid phase peptides is then probed with test antibodies and bound antibody is detected using enzyme-labeled secondary antibody. Methods for mapping linear epitopes are known in the art (see, e.g., Harlow and Lane, supra).

Alternatively, antigenic epitopes can be mapped by competition assay. Competition assay is a widely used method for determining if two antibodies are able to bind independently to the same protein antigen or whether their binding sites on the same protein overlap in such a way that both are not able to bind to the antigen at the same time. (see, e.g., Harlow and Lane, supra).

C. Humanized Antibodies and Chimeric Antibodies

Techniques for humanizing antibodies involve selecting the complementarity determining regions (CDRs), i.e., the antigen binding loops, from a donor monoclonal antibody, and grafting them onto a human antibody framework of known three dimensional structure (see, e.g., WO98/45322; WO 87/02671; U.S. Pat. No. 5,859,205; U.S. Pat. No. 5,585,089; U.S. Pat. No. 4,816,567; EP Patent Application 0173494; Jones, et al. (1986) Nature 321:522; Verhoeyen, et al., (1988) Science 239:1534 Riechmann, et al. (1988) Nature 332:323; and Winter & Milstein, (1991) Nature 349:293).

The positions of the CDR's and hence the positions of the framework regions of the human heavy chain and light chains are determined using definitions that are standard in the art. For example, framework regions and antigen binding loop regions may be identified using a number of antigen binding loop definitions such as those by Kabat, Chothia, IMGT (Ruiz, et al., Nucleic Acids Res. 28:219-221 (2000); and Lefranc, Nucl. Acids Res. 29:207-9 (2001)), AbM (Martin et al., Proc. Natl. Acad. Sci. USA, 86:9268-9272, (1989); Martin et al, Methods Enzymol. 203:121-153 (1991); Pedersen et al, Immunomethods 1:126 (1992); and Rees et al, In Sternberg M. J. E. (ed.), Protein Structure Prediction. Oxford University Press, Oxford, 141-172, (1996)), and contact (MacCallum et al., J. of Mol. Biol. 262:732-745 (1996)).

Human framework sequences can be obtained by the skilled artisan using well known techniques, e.g., using phage display libraries (see, e.g., Sastry et al., Proc Natl Acad Sci USA 86:5728-5732, 1989; McCafferty et al., Nature 348:552-554, 1990; Marks et al., J Mol Biol 222:581-597, 1991; Clackson et al, Nature 352:624-628, 1991; and Barbas et al., Proc Acad Sci USA 88:7978-7982, 1991) to isolate human V_{H} and V_{L} sequences, for example, corresponding to the B-cell repertoire of one or more individuals. The sequences are determined using standard technology.

V_{H} and V_{L} amino acid sequences are then aligned with a donor antibody, e.g., the antibody with the idiotype which specifically binds to the anti-idiotypic antibody used for screening, to select frameworks for humanizing the donor antibody. In brief, the heavy and light chain variable sequences of a donor antibody of interest, e.g., the murine monoclonal antibody 9F12 or 10F2, are aligned with uncharacterized human heavy and light chain sequences using e.g., the Abcheck software, e.g., available at http://www.bioinf.org.uk/abs/(e.g., Martin, A. C. R. (1996) Accessing the Kabat antibody sequence database by computer. PROTEINS: Structure, Function and Genetics, 25, 130-133). The software aligns the provided sequence to a consensus sequence to map it to the Kabat numbering system. In an additional step, the aligned sequence is scanned against the Kabat database. The human sequences that have sequence identity of at least about 70% or greater are selected for candidate framework sequences for humanization. It is known that the function of an antibody is dependent on its three dimensional structure, and that amino acid substitutions can change the three-dimensional structure of an antibody. However, the antigen binding affinity of a humanized antibody can be increased by mutagenesis based upon molecular modeling (Riechmann, L. et al., Nature 332:323-227 (1998); Queen, C. et al., Proc. Natl. Acad. Sci. USA 6:10029-20033 (1989)). Thus, sequences selected for humanization are analyzed to determine important framework residues that can be backmutated to the donor sequence to obtain stable antibodies that bind to the same epitope as the donor antibody with a comparable affinity.

Chimeric antibodies are distinguished from humanized antibodies primarily in that the framework region is not derived from a human framework. Chimeric antibodies may be constructed by methods similar to those described above for the production of humanized antibodies.

D. Labeled Antibodies

Antibodies of the present invention may optionally be covalently or non-covalently linked to a detectable label. Detectable labels suitable for such use include any composition detectable by spectroscopic, photochemical, biochemical, immunochemical, electrical, optical or chemical means. Useful labels in the present invention include magnetic beads (e.g. DYNABEADS), fluorescent dyes (e.g., Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 635, Alexa Fluor 647, Alexa Fluor 660, Alexa Fluor 680, Alexa Fluor 700 and Alexa Fluor 750 dyes, fluorescein isothiocyanate, Texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g. polystyrene, polypropylene, latex, etc.) beads.

The procedure for attaching an effector molecule to an antibody will vary according to the chemical structure of the moiety to be attached to the antibody. Polypeptides typically contain a variety of functional groups; e.g., carboxylic acid (COOH), free amine (—NH2) or sulfhydryl (—SH) groups, which are available for reaction with a suitable functional group on an antibody to result in the binding of the effector molecule.

Alternatively, the antibody is derivatized to expose or to attach additional reactive functional groups. The derivatization may involve attachment of any of a number of linker molecules such as those available from Pierce Chemical Company, Rockford Ill.

The linker is capable of forming covalent bonds to both the antibody and to the effector molecule. Suitable linkers are well known to those of skill in the art and include, but are not limited to, straight or branched-chain carbon linkers, heterocyclic carbon linkers, or peptide linkers. Where the antibody and the effector molecule are polypeptides, the linkers may be joined to the constituent amino acids through their side groups (e.g., through a disulfide linkage to cysteine). However, in a preferred embodiment, the linkers will be joined to the alpha carbon amino and carboxyl groups of the terminal amino acids.

Means of detecting such labels are well known to those of skill in the art. Thus, for example, radiolabels may be detected using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

E. Detecting Antibody-Antigen Complex in a Sample

Once produced, conformation specific antibodies may be used in virtually any assay format that employs antibodies to detect antigens. Design of the immunoassays is subject to a great deal of variation, and many formats are known in the art. Protocols may, for example, use solid supports, or immunoprecipitation. Most assays involve the use of labeled antibody or polypeptide; the labels may be, for example, enzymatic, fluorescent, chemiluminescent, radioactive, or dye molecules, as discussed in detail above. Assays which amplify the signals from the immune complex are also known; examples of which are assays which utilize biotin and avidin, and enzyme-labeled and mediated immunoassays, such as ELISA assays.

1. ELISA

In ELISA assays, a biological sample to be tested for the presence of a specific conformation of a protein is immobilized onto a selected surface, for example, a surface capable of binding proteins, such as the wells of a polystyrene microtiter plate. The solid support is reacted with the sample, under suitable binding conditions such that the molecules are sufficiently immobilized to the support. Sometimes, immobilization to the support can be enhanced by first coupling the antigen and/or antibody to a protein with better solid phase-binding properties. Suitable coupling proteins include, but are not limited to, macromolecules such as serum albumins including bovine serum albumin (BSA), keyhole limpet hemocyanin, immunoglobulin molecules, thyroglobulin, ovalbumin, and other proteins well known to those skilled in the art. Other reagents that can be used to bind molecules to the support include polysaccharides, polylactic acids, polyglycolic acids, polymeric amino acids, amino acid copolymers, and the like. Such molecules and methods of coupling these molecules to antigens, are well known to those of ordinary skill in the art. See, e.g., Brinkley, M. A. (1992) Bioconjugate Chem. 3:2-13; Hashida et al. (1984) J. Appl. Biochem. 6:56-63; and Anjaneyulu and Staros (1987) International J. of Peptide and Protein Res. 30:117-124.

After washing to remove incompletely adsorbed antigens, a nonspecific protein such as a solution of bovine serum albumin (BSA) that is known to be antigenically neutral with respect to the test sample may be bound to the selected surface. This allows for blocking of nonspecific adsorption sites on the immobilizing surface and thus reduces the background caused by nonspecific bindings of antisera onto the surface.

The immobilizing surface is then contacted with the conformation specific antibodies of the invention, in a manner conducive to immune complex (antigen/antibody) formation. The mixture is then allowed to incubate for from 2 to 24 hours, at temperatures such as of the order of about 25° C. to 37° C. Following incubation, the conformation specific antibody-contacted surface is washed to remove non-immunocomplexed material. The washing procedure may include washing with a solution, such as PBS/Tween or a borate buffer.

Following formation of specific immunocomplexes between the conformation specific antibody and the affixed test sample, and subsequent washing, the occurrence, and even amount, of immunocomplex formation may be determined by subjecting the immunocomplex to a second antibody having specificity for the conformation specific antibody, as is known in the art. To provide detecting means, the second antibody may have an associated activity such as an enzymatic activity that will generate, for example, a color development upon incubating with an appropriate chromogenic substrate. Quantification may then be achieved by measuring the degree of color generation using, for example, a spectrophotometer.

### VII. Use of Conformation Specific Protein Binding Domains or Antibodies to Identify Compounds that Induce the Adoption of Specific Protein Conformations

Conformation specific protein binding domains or antibodies may be used in screens of libraries of compounds to identify compounds which induce the adoption of specific conformational states by a protein. In general, a screen for compounds that induce the adoption of particular conformations will involve contacting a protein of interest, either purified, partially purified, or in an intact cell, with a member of a library of compounds. As discussed above, frequently the protein will be immobilized onto a surface or contained within a well of a multi-well plate. Whether a test compound has induced the adoption of a particular conformational state in the protein can then be determined by applying the conformation specific protein binding domains or antibodies of the invention to the protein in the presence of the test compound. A small molecule that causes the protein to assume a specific conformation recognized by the protein binding domain or antibody will result in increased antibody binding to the protein as compared to a control sample which is identical except that it lacks a test compound.

The compounds to be tested can be any small chemical compound, or a macromolecule, such as a protein, sugar, nucleic acid or lipid. Typically, test compounds will be small chemical molecules and peptides. Essentially any chemical compound can be used as a test compound of the invention, although most often compounds that are soluble in aqueous or organic (especially DMSO-based) solutions are used. Assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries are well known to those of skill in the art. Such combinatorial chemical libraries include, but are not limited to, peptide libraries (see, e.g., U.S. Pat. No. 5,010,175, Furka, Int. J. Pept. Prot. Res., 37:487-493 (1991) and Houghton et al., Nature, 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., PNAS USA, 90:6909-6913 (1993)), vinylogous polypeptides (Hagihara et al., J. Amer. Chem. Soc., 114:6568 (1992)), nonpeptidal peptidomimetics with glucose scaffolding (Hirschmann et al., J. Amer. Chem. Soc., 114:9217-9218 (1992)), analogous organic syntheses of small compound libraries (Chen et al., J. Amer. Chem. Soc., 116:2661 (1994)), oligocarbamates (Cho et al., Science, 261:1303 (1993)), and/or peptidyl phosphonates (Campbell et al., J. Org. Chem., 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

The assays can be solid phase or solution phase assays. In the high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 96 modulators. If 1536 well plates are used, then a single plate can easily assay from about 100- about 1500 different compounds. It is possible to assay many plates per day; assay screens for up to about 6,000, 20,000, 50,000, or 100,000 or more different compounds is possible using the integrated systems of the invention.

### EXAMPLES

The following examples are provided to illustrate, but not to limit the claimed invention.

### Example 1

### Expression, Purification, and Assays for Inflammatory Capases

Wild type and mutants of caspase-1 are readily cloned and expressed in the pRSET vector (Invitrogen, Carlsbad, Calif.). Typical refolding and purification yields about 2 mg of pure heterodimeric protein using methods developed in our laboratory (see Scheer, J. M. et al., Proc Natl Acad Sci USA, 103(20):7595-7600 (2006)). For disulfide screening a variant of caspase-1 was produced with four alanine mutations that remove other surface cysteines, three in the small subunit (C362A, C364A, C397A) and one in the large subunit (C285A). The three alanine mutations in the small subunit facilitate deconvolution of the mass spectrometry data by leaving only the allosteric cysteine, and these do not significantly impact catalytic activity. The C285A active site mutation in the large subunit was made to prevent any autocatalytic degradation during the disulfide trapping screen that would complicate the mass spectrum. We have solved the x-ray structure of the triple mutant enzyme in complex with an active site inhibitor and it shows an identical conformation to the inhibitor bound wild type enzyme (data not shown).

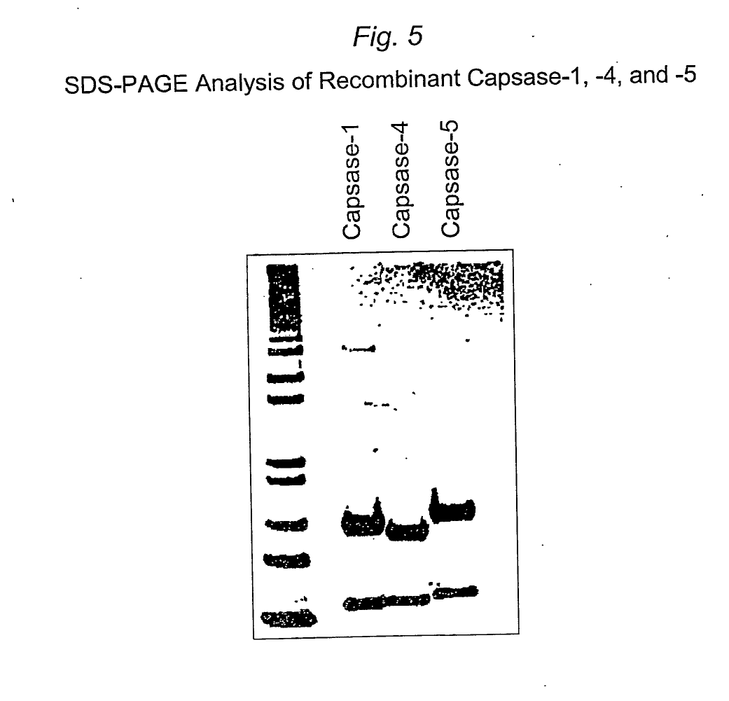

We have cloned both the small and large subunits of caspase-4 and caspase-5 in the pRSET bacterial expression vector. All four subunits have been expressed to greater than 10 mg per liter in E coli and have subsequently been refolded and purified by ion exchange chromatography (FIG. 5). Both caspase-4 and caspase-5 recombinant enzymes display wild type kinetics (data not shown). Initial crystallographic trials have been performed on caspase-4 in complex with an active site inhibitor bound. One of these conditions has produced crystals that may be suitable for x-ray diffraction.

### Example 2

### Identification of Disulfide-Trapped Compounds to Caspase-1

The disulfide-trapping screens are run in a redox buffer to ensure facile thiol-disulfide exchange (see Erlanson, D. et al., Proc. Natl. Acad. Sci. USA, 97(17):9367-9372 (2000)). The reductant is β-ME and the disulfides come from the fragment compounds. The stringency of the screen depends in part on the concentration of reductant (β-ME) and the thiol on the protein. A convenient way to determine the suitable reductant concentration is to titrate the protein with β-ME in a fixed concentration of oxidized (β-ME (generally 0.1 mM) and determine the time it takes to equilibrate with labeling on the protein (generally about 30 min at room temperature). One also determines the concentration of reduced β-ME needed for 50% labeling which sets the reduced β-ME concentration to be used in the fragment screen (generally from 0.1 to 1 mM). We have found it is most efficient to screen in pools of 10 compounds (50 μM per compound). Pools are constructed so that each compound in the pool differs from the others by 5 mass units which is easily resolved by mass spectrometry. The mass of the hit compound in the pool is readily identified by taking the difference in molecular weight of the conjugated and non-conjugated protein. Hits found in the first screen are then tested as individual purified compounds to confirm the result. In our experience the hit reconfirm rate is about 85%. All primary hit compounds are tested on the wild-type form of caspase-1 (“non-scrubbed”) to ensure that single site labeling is observed in the face of all surface thiols. Hits are defined as reconfirmed compounds that conjugate to a level of >25% to the specific thiol. In general the hit rate from disulfide trapping is in the range 0.1 to 1% so that labeling patterns rarely involve two sites or two compounds and provide unambiguous interpretation.

The screening construct of caspase-1 described above was used in a disulfide trapping screen of 8,000 compounds from the Sunesis disulfide compound library (see Erlanson, D. A. et al., Annu. Rev. Biophys. Biomol. Struct., 33:199-223 (2004)). The caspase-1 variant (5 μM) in a buffer containing 50 mM HEPES pH 7.5, 200 mM NaCl, 5% mM KCl, and 0.1 mM β-ME was incubated for one hr with pools containing 10 compounds each at a final concentration of 50 μM per compound (in 2% DMSO). After incubation, the reactions were quenched with 5 μL of 1 N HCl to stop the disulfide exchange reaction, and analyzed by high throughput mass spectrometry. Pools containing putative hits were deconvoluted as discrete compounds in a second screen that yielded 57 reconfirmed hits to the small subunit allosteric cysteine. The hits were tested for inhibitory activity in an enzyme assay using the fluorescent substrate Ac-WEHD-AFC (Axxora, San Diego, Calif.). All were functional inhibitors of enzyme activity and inhibition was fully reversed by reduction.

Six of the compounds with highest conjugation in the initial screen were characterized in greater detail. First we determined the concentration of compound needed to cause 50% labeling at a fixed concentration of the reductant β-ME, termed dose response₅₀ or DR₅₀. This provides an estimate of the how well the compound binds to the protein under strongly reducing conditions in a cell. A second characterization involves fixing the concentration of the hit compound and determining the concentration of β-ME at which 50% of the compound remains bound, termed β-ME₅₀. This provides further evidence that the compound has a high reduction potential and that the conjugation is fully reversible. Positive hits typically have DR₅₀ values (at 1 mM β-ME) of 1-50 μM, and β-ME₅₀ values of 2-20 mM at 50 μM compound. Since we are using the disulfide hits directly as functional probes, these values are directly useful for comparisons of the potencies of the compounds in subsequent biochemical and cell-based assays. All six showed very good DR₅₀ and β-ME₅₀ values ranging from 12-24 μM and 8-24 mM, respectively (Table 1).

TABLE 1
DR₅₀ and β-ME₅₀ for selected cystamine-
disulfide compounds for inhibiting caspase-1.

| compound | | DR₅₀ | β-ME₅₀ |
|---|---|---|---|
| no. | structure | (μM) | (mM) |
| 3 | 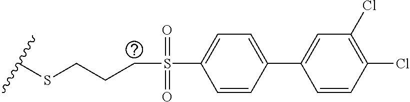 | 12 | 21 |
| 4 | 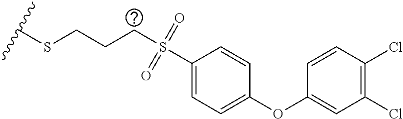 | 9.5 | 12 |
| 11 | 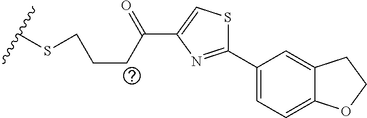 | 12 | 7.2 |
| 20 | 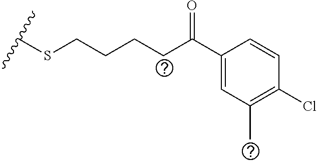 | 9.0 | 24 |
| 32 | 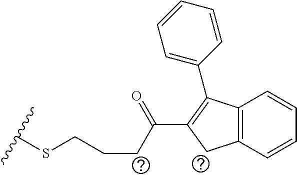 | 12 | 10 |
| 34 | 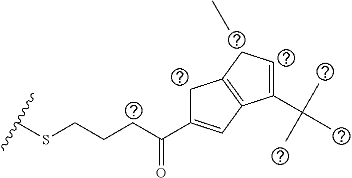 | 24 | 17 |

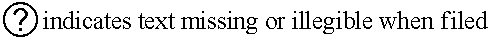

### Example 3

### Structure of Compound #34 in Complex with Caspase-1

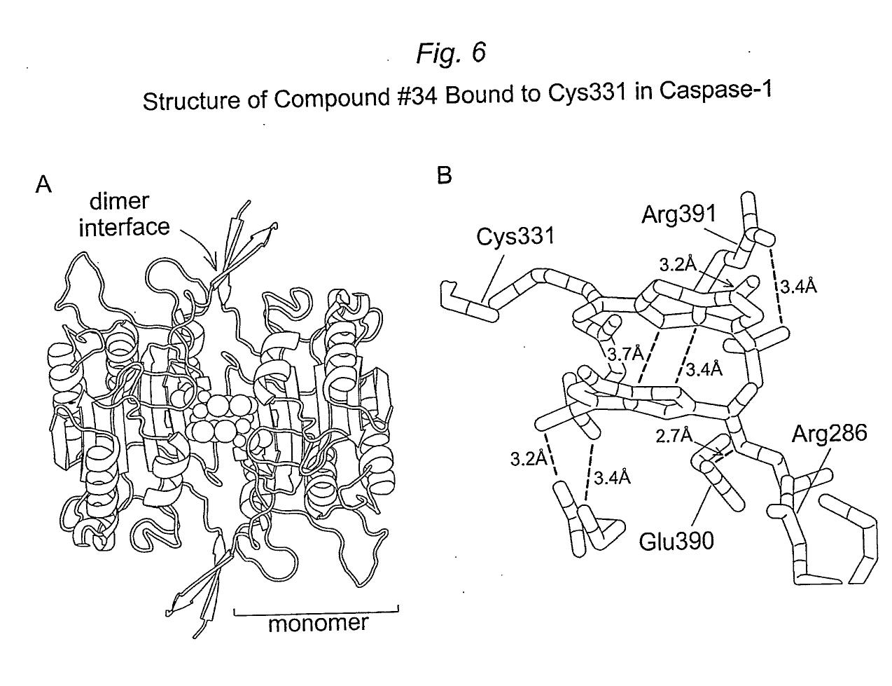

We have solved the x-ray structure of caspase-1 in complex with a disulfide bound allosteric site inhibitor, compound #34 (see Scheer, J. M. et al., Proc Nall Acad Sci USA, 103(20):7595-7600 (2006)). FIG. 6A shows the position of two molecules of the thiophene-pyrazole compound in the cavity at the dimer interface in caspase-1. The compound extends from Cys331 across the dimer interface in a trans-like configuration and disrupts a hydrogen-bonding network at the dimer interface (FIG. 6B). Notably the thiophene-pryazole ring shears a salt-bridge interaction between Arg286 and Glu390 and its amide is within H-bond distance of the displaced Glu390.

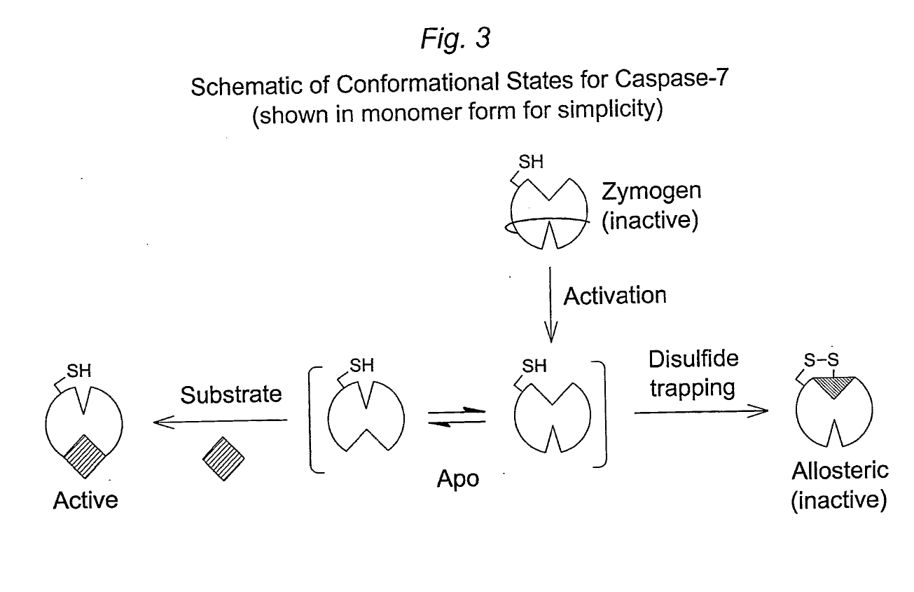

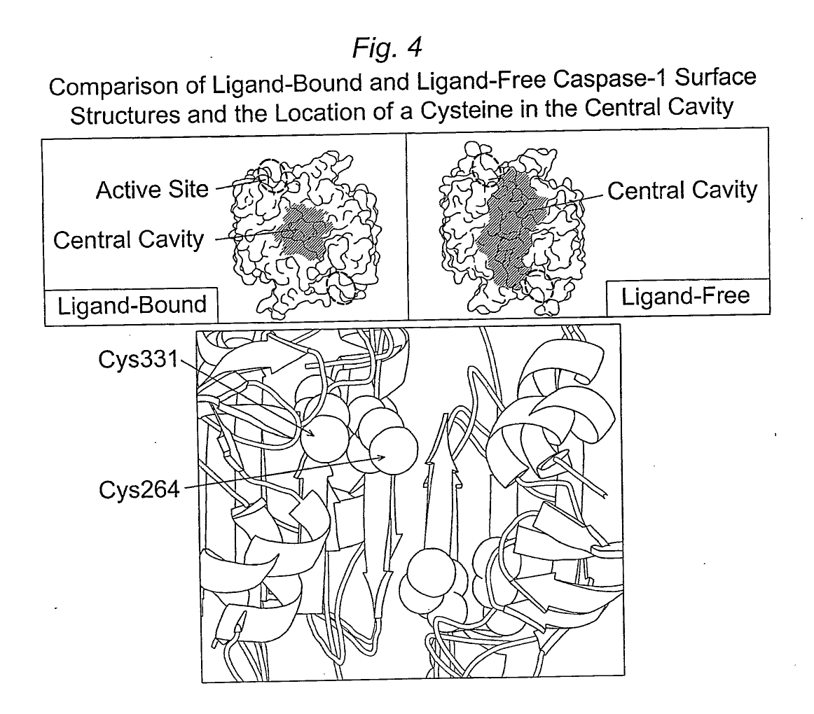

Structural overlays show the allosterically inhibited form of caspase-1 is virtually identical to that of the apo-form of the enzyme. These data provide strong support for allosteric inhibitors trapping a natural form of the enzyme. We have previously found that allosteric inhibitors in a similar position in caspase-7 trapped a conformation very close to that of Pro-caspase-7 (see Hardy, J. et al., Proc Nall Acad Sci U S A, 101(34):12461-12466 (2004)). Thus the allosteric inhibitors trap “off” states of the caspases by binding to a similar site at the dimer interface. The combination of these structural studies on both caspase-1 and -7 provides evidence to support a model for dynamic activation of these enzymes as shown in FIG. 3.

### Example 4

### Mutational Analysis of Caspase-1 Allosteric Circuitry

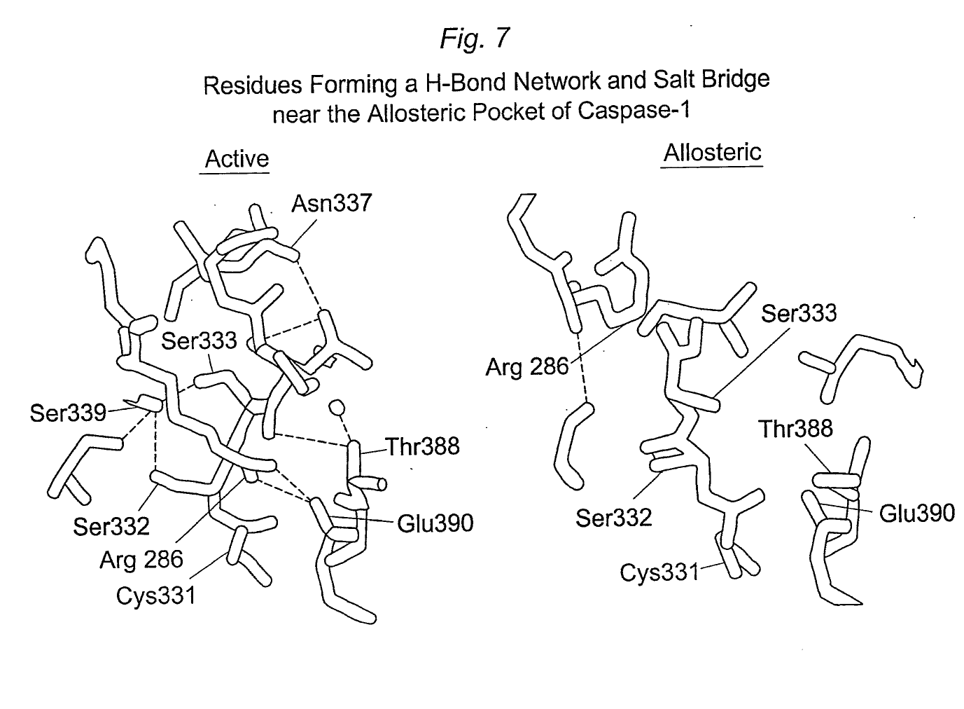

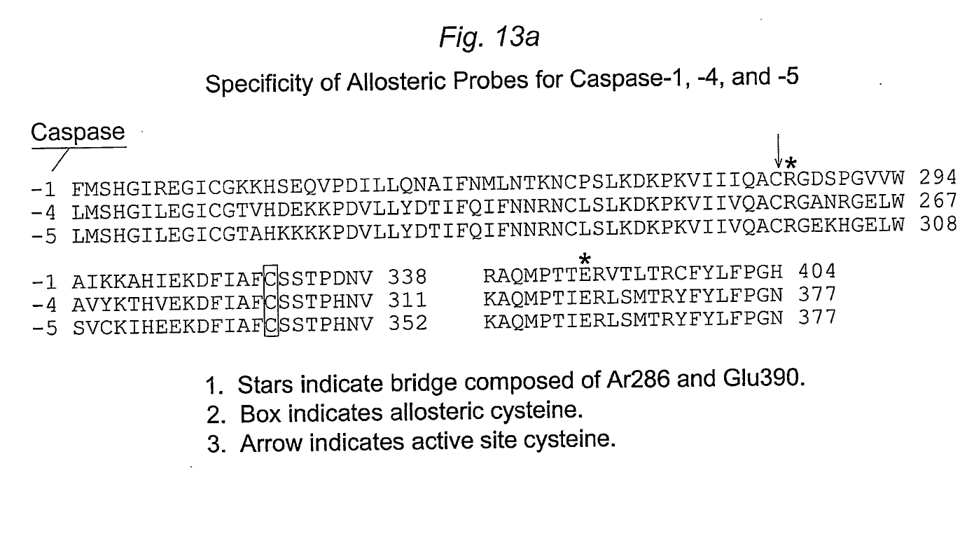

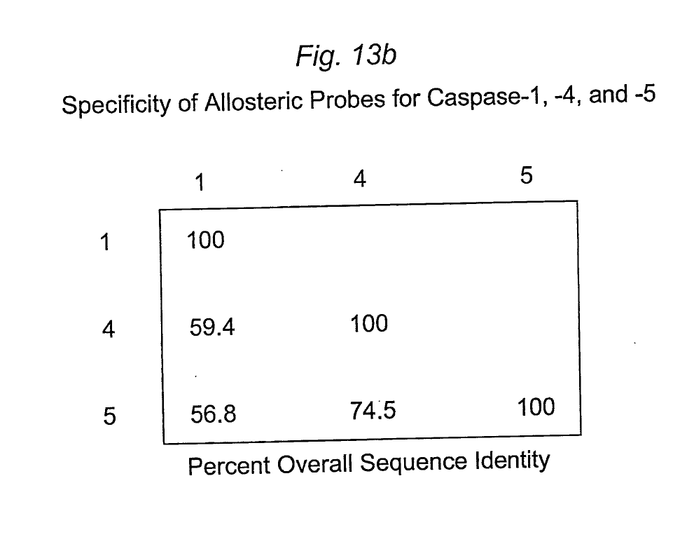

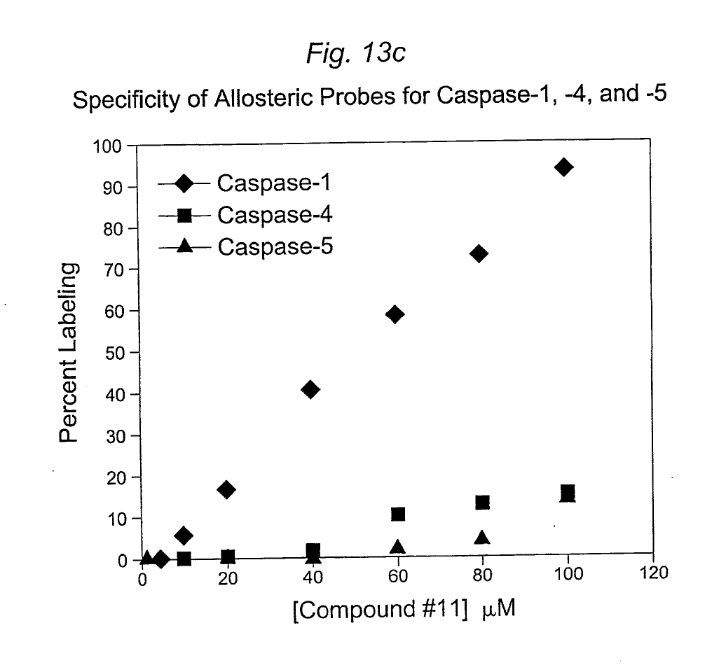

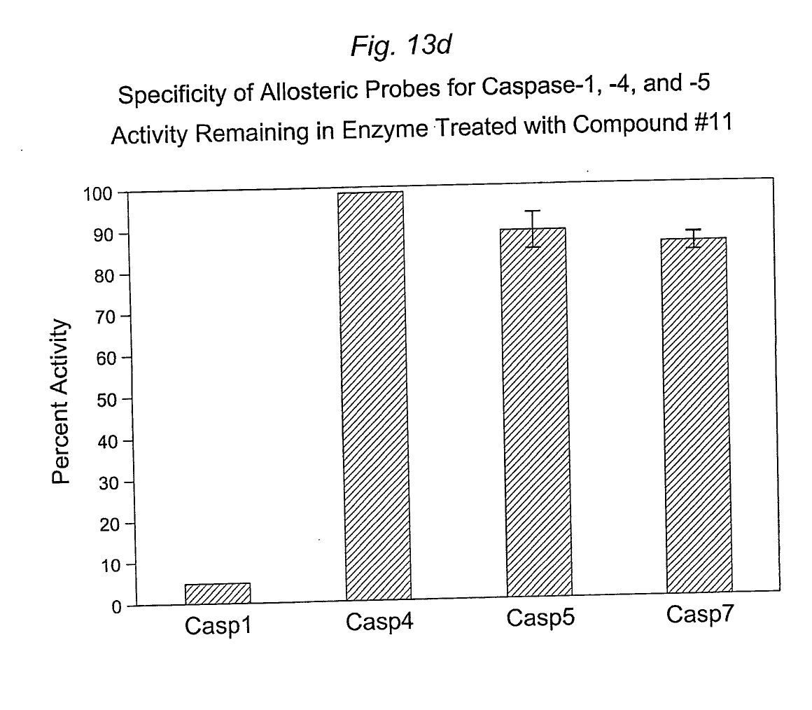

Several lines of evidence suggest that the active sites of caspase-1 are functionally coupled and this coupling is mediated by a “circuit” of residues that run from one active site through the allosteric site to the second active site. The allosteric inhibitors directly disrupt this circuit by breaking the Arg286-Glu390 salt bridge. These residues and others form an H-bonding network (FIG. 7) that is well conserved among the inflammatory caspases (FIG. 13). Binding of the active site inhibitor z-VAD-FMK and the allosteric inhibitors are mutually exclusive even though their binding sites do not overlap (see Scheer, J. M. et al., Proc Natl Acad Sci USA, 103(20):7595-7600 (2006)). We recently discovered caspase-1 shows positive cooperativity and a Hill coefficient of 1.5, indicating functional coupling of the two active sites. To better understand the residues in caspase-1 that are functionally responsible for mediating the coupling and conformational switch we employed alanine-scanning mutagenesis (see Cunningham, B. and J. Wells, Science, 244:1081-1085 (1989)). This approach has been effective for identifying functional “hot-spots” in protein interfaces (see Clackson, T. and J. Wells, Science, 267:383-386 (1995)).

By inspecting the differences between the active and allosterically inhibited form of the protein we can identify many residues that have shifted position between the two sites (see FIG. 7). Residues whose side chain positions change significantly (>3-5 Å) relative to the active enzyme and sit between the two sites were selected for alanine-scanning mutational studies (listed in Table 2). The mutated side chains fall into two categories: polar side chains which are important in forming a hydrogen bonding network in the active conformation, and other side chains that involved in the conformational flexibility of loop regions that change position between the allosteric and active conformations. QuikChange mutagenesis (Invitrogen, Carlsbad, Calif.) was used to produce single site alanine mutants for each of these residues in either the p10 or p20 subunit. These were expressed, purified, and refolded as described earlier. The presence of a single site mutation in each of the refolded caspase-1 constructs was verified by mass spectrometry, and then analyzed by Michaelis-Menten kinetics.

### The H-Bonding Network:

There is a hydrogen-bonding network that connects the active site with the central cavity (see FIG. 7). When compound #34 severs the Arg286-Glu390 salt bridge it allows the side-chain of Arg286 to flip into the active site (see FIG. 6). We examined whether this salt-bridge was critical for stabilizing the active form of the enzyme by disrupting the salt bridge by directed mutagenesis. The E390A mutation produced a dramatic 290-fold reduction in catalytic efficiency (k_{cat}/K_{M}) which was distributed as a 7-fold decrease in k_{cat} and a 40-fold increase in K_{M} (Table 2). Similar effects were seen for the R286A mutation. Given that acylation is the rate-limiting step in nucleophilic protease mechanisms (see Gutfreund et al., Biochemical Journal, 63: 656-661 (1956)), the simplest interpretation is that breaking this non-substrate contact residue indirectly affects both the substrate binding and catalytic step. In fact, the x-ray structure shows compound #34 disrupts both the loops responsible for substrate binding and the alignment of the catalytic cysteine and histidine (see Scheer, J. M. et al., Proc Natl Acad Sci USA, 103(20):7595-7600 (2006)).

Of the mutations of side chains involved in the hydrogen-bonding network, the R341A mutant had the largest effect which was symmetrically distributed both by increasing K_{M} and decreasing the k_{cat}. Arg341 makes a salt bridge with the P1 aspartate of the substrate so is likely responsible for both substrate recognition and stabilization of the substrate-bound active conformation. The S332A mutant showed a significant decrease in k_{cat} with no change in the K_{M}. This suggests that the hydrogen bonding interactions made by the hydroxyl side chain are important for stabilizing the active site and catalytic turnover, but not for binding of substrate. Four of the other mutants in the hydrogen bonding network, S333A, T334A, D336A, and N337A showed small, increases (−2-fold) in the K_{M} and also an increase in the Hill coefficient. The T388A mutant appeared to have the smallest effect, with little change in the k_{cat}, K_{M}, or Hill coefficient. Thus, the integrity of the H-bonding network is functionally critical for activity, and the central salt-bridge and Arg341 are key hot-spots.

TABLE 2
Mutations probing the allosteric circuitry of caspase-1.

| | | | | k_{cat}/K_{M}, | | |
|---|---|---|---|---|---|---|
| | | K_{M}, | k_{cat}, | M⁻¹ · | Ratio, | |
| Residue | Rationale | μM | sec⁻¹ | sec⁻¹ | k_{cat}/K_{M} | n_{HILL} |
| WT | | 4.8 | 0.51 | 1.1 × 10⁵ | 1.0^{†} | 1.4 |
| Side chains involved in H-bonding network | | | | | | |
| Arg | H-bond to Ser333 carbonyl, and salt | 370 | 0.17 | 4.6 × 10² | 240 | 1.6 |
| 286 | bridge to Glu390 in active; no | | | | | |
| | interactions in allosteric bound | | | | | |
| Ser 332 | H-bond to Ser339 side chain hydroxyl | 3.8 | 0.024 | 6.3 × 10³ | 18 | 1.3 |
| | in active structure; H-bond only to its | | | | | |
| | own carbonyl in allosteric | | | | | |
| Ser 333 | H-bond to Ser339 side chain hydroxyl | 11 | 0.35 | 3.3 × 10⁴ | 3.4 | 1.8 |
| | in active structure; no H-bonds in | | | | | |
| | allosteric | | | | | |
| Thr 334 | H-bond to Asn337 in active; no | 13 | 0.40 | 3.2 × 10⁴ | 3.5 | 1.80 |
| | interactions in allosteric | | | | | |
| Asp | Salt bridge interaction with Arg240 in | 9.1 | 0.50 | 5.5 × 10⁴ | 2.0 | 1.8 |
| 336 | active, helps keep Arg in contact with | | | | | |
| | protein; engages in salt bridge | | | | | |
| | interaction with Arg383 in allosteric | | | | | |
| | structure | | | | | |
| Asn | H-bond to Thr334 side chain, | 9.1 | 0.24 | 2.6 × 10⁴ | 4.2 | 1.9 |
| 337 | backbone amide of Asn337 and | | | | | |
| | G1y391 in active; interacts only with | | | | | |
| | it's backbone amide and carbonyl in | | | | | |
| | allosteric | | | | | |
| Arg | H-bond distance to Thr180 carbonyl | 210 | 0.02 | 9.4 × 10¹ | 1200 | 1.3 |
| 341 | and amide in active; no contacts in | | | | | |
| | allosteric | | | | | |
| Thr 388 | H-bond to Ser333 carbonyl and | 5.8 | 0.24 | 4.2 × 10⁴ | 2.7 | 1.5 |
| | Met386 carbonyl in active structure; H- | | | | | |
| | bond with Glu390 in allosteric bound | | | | | |
| Glu 390 | Arg286 salt bridge in active structure; | 180 | 0.07 | 3.9 × 10² | 290 | 1.0 |
| | H-bond with Thr388 side chain and | | | | | |
| | Arg391 amide in allosteric | | | | | |
| SSide chains involved in conformational | | | | | | |
| flexibility | | | | | | |
| Gly 287 | Moving loop adjacent to active site | 200 | 0.0066 | 3.5 × 10¹ | 3200 | 1.0 |
| | cysteine | | | | | |
| Pro 335 | Part of moving loop region | 7.9 | 0.57 | 7.3 × 10⁴ | 1.5 | 1.6 |
| Pro 343 | Located on loop turn that changes | 2.3 | 0.58 | 2.6 × 10⁵ | 0.4 | 1.5 |
| | position in active and allosteric | | | | | |
| | conformations | | | | | |

*Standard errors within X % of reported values;
^{†}Ratio of k_{cat}/K_{M} relative to wildtype

Hinge Points that Mediate Conformational Changes:

The flipping of Arg286 between the active and inactive states appears mediated by Gly287 located on a moving loop adjacent to the active site cysteine (Cys285). The dihedral angles (φ and ψ) of Gly287 in the allosteric structure show angles of approximately −83° and 56°, respectively. These are sterically allowed for alanine. However in the active structure, this loop undergoes a large conformational change that kinks the polypeptide backbone at this position producing φ and ψ angles of approximately 149° and -174°. The dihedral angles in the active conformation are allowed for glycine, but not for alanine. The G287A mutant resulted in a massive 3100-fold decrease in catalytic efficiency that was about evenly distributed for effects on K_{M} and k_{cat}. We conclude that the G287A mutant is locked in the inactive state and prevented from assuming the active conformation by steric hindrance from the alanine.

In contrast, the P343A mutant showed an increase in catalytic turnover (k_{cat}) with little change in binding (K_{M}). Pro343 is located on a loop forming part of the active site, and this loop changes position when caspase-1 goes from the apo to active conformation. Mutation of this “gate keeper” side chain likely increases flexibility of this loop, making it easier for caspase-1 to adopt the active conformation. Mutation of another proline at position 335, also located on a moving loop region, has little effect on any of the kinetic values of caspase-1. Thus, not all praline residues are important. In summary, we have identified several residues located in the core of the protein outside of the enzyme's active site that are important for catalytic activity and some mediate conformational change between the allosteric and active conformation of caspase-1.

### Example 5

### Positive Cooperativity in Caspase-1

We recently discovered that caspase-1 shows positive cooperativity with a Hill coefficient of 1.5 (see Scheer, J. M. et al., Proc Natl Acad Sci USA, 103(20):7595-7600 (2006)). The Hill coefficient gives the extent of coupling between the active sites. To determine to what extent mutations in the allosteric circuit impact coupling we measured their Hill coefficients (Table 2). Most of the mutated residues that severely impacted catalytic efficiency (E390A, R341A and G287A) showed significantly reduced Hill coefficients suggesting they uncoupled the active sites. Mutations with less impact on catalytic efficiency had little impact on the Hill coefficient. The one exception was for R286A which caused a 240-fold reduction in catalytic efficiency but almost no change in Hill-coefficient. This suggests this residue which sits next to the catalytic Cys285 affects transition-state stabilization and has less of a role in inter-site coupling.

Although the Hill plot provides evidence for positive cooperativity, the Hill coefficient only reports on the degree of subunit interaction and can not go higher than the number of cooperative subunits (in this case two). It does not provide information on the functional impact on the second active site after binding occurs in the first site. This raised the question as to what the activity of a single active site is when the other active site is locked in the active conformation.

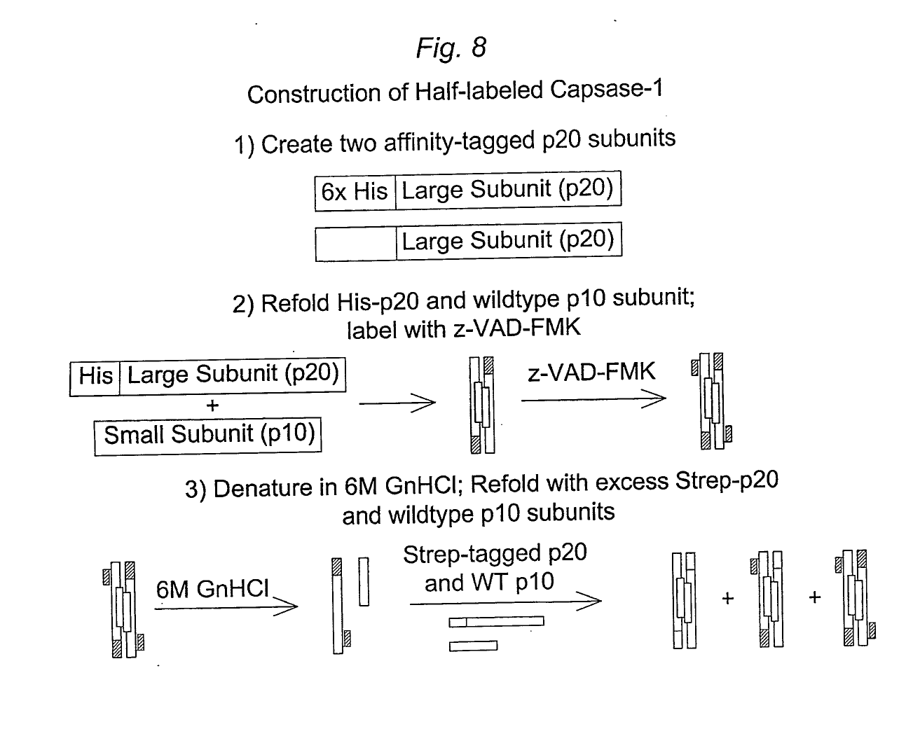

In order to begin to answer this question, we devised a method for reliably locking one of the active sites in the on state. From x-ray analysis, we know that the structures of caspase-1 are virtually the same whether the active site is occupied by various non-covalent or covalent inhibitors (see Romanowski, M. J. et al., Structure (Camb), 12(8):1361-71 (2004)). Thus, to a first approximation it was possible to lock the “on” state by labeling with the active site inhibitor z-VAD-FMK. However, we needed to create a single-site labeled enzyme. This was accomplished by creating two affinity-tagged large (p20) subunits, the first with a 6×His tag and the other with a Strep-tag (FIG. 8). The Strep-tag is an eight amino acid peptide with highly selective binding properties to the streptavidin variant Strep-Tactin (IBA GmbH, Germany). These tagged p20 subunits were then refolded separately with a wild-type p10 small subunit and purified by cation exchange chromatography as described earlier. These His-tagged and Strep-tagged “homodimers” were refolded and exhibited catalytic activity similar to wild-type caspase-1.

The His-tagged caspase-1 homodimer was then labeled with the irreversible active site inhibitor z-VAD-FMK to lock the active form of the enzyme (FIG. 8). This resulted in labeling of both active sites in the His-tagged homodimers as verified by mass spectrometry and complete inhibition of catalytic activity. The labeled His-tagged caspase-1 was then denatured in 6M guanidine and refolded in the presence of the Strep-tagged p20 subunit and excess p10 subunit. This resulted in the generation of three caspase-1 species: (1) an unlabeled Strep-tagged homodimer; (2) a singly labeled His/Strep-tagged hybrid “heterodimer;” (3) and a doubly labeled His-tagged homodimer. These three species could be resolved using cation exchange chromatography due to the charged residues present in the His and Strep tags. The purification of heterodimer caspase-1 with only one active site bound with the active site inhibitor was verified by double affinity purification and mass spectrometry.

Using the singly-labeled, hybrid caspase-1 construct, we were able measure the impact on enzymatic activity of locking one active site of the caspase-1 dimer in the active conformation. The protease was analyzed by Michaelis-Menten kinetics using the Ac-WEHD-AFC fluorescent substract. The labeled hybrid construct had a 10-fold increase in catalytic efficiency (per active subunit) that was due to a 20-fold increase in k_{cat} and a 2-fold increase in Km. This data shows that forcing one active site of the caspase-1 dimer into the active conformation greatly enhances the activity of the other active site, reinforcing the idea that the catalytic mechanism of this protease is highly cooperative.

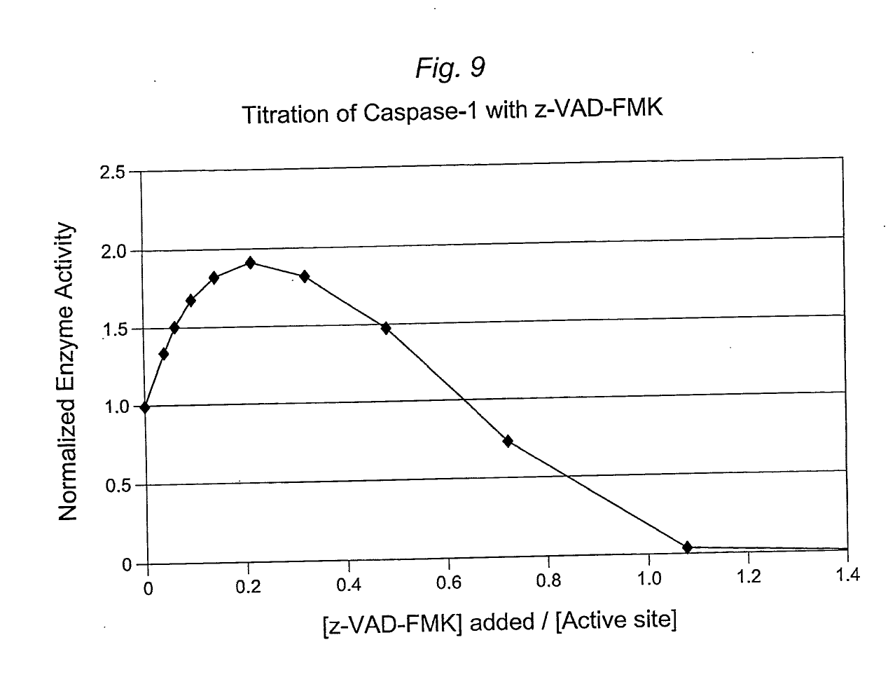

This 10-fold increase in catalytic efficiency for the half-labeled hybrid suggested we may observe some activation for wild-type enzyme when partially inhibited by z-VAD-FMK. To test this, wild-type caspase-1 was titrated with inhibitor and found to produce a 2-fold activation when one reaches 0.2 equivalents of z-VAD-FMK. Further addition lead to complete inhibition when approaching one equivalent of inhibitor (FIG. 9). If the inhibitor labeled in a non-cooperative fashion we would expect that it would reach an optimum upon addition of about 0.5 equivalents and would cause a 5-fold activation as half of the sites are inactivated. The fact this maxima is below 0.5 equivalents and is less than 5-fold probably reflects that labeling is cooperative too. Moreover, the mutations which lower the Hill-coefficient presumably by disrupting active site coupling do not show this partial activation. Thus, we have developed a second assay for evaluating the active site coupling.

Positive cooperativity has not been reported for other endopeptidases. In the case of caspase-1, it could provide an additional level of control since caspase-1 would become more active at high concentrations when assembled into inflammasomes. Since the processing by the other caspases are predicted to be driven from association with inflammosomes, we hypothesize that both caspase-4 and -5 show positive cooperativity and inhibitor activation.

### Example 6

### Generation of Conformation Specific Antibodies to the Allosteric and Active Conformations of Caspase-1 by Phage Display to Probe these Forms In Vitro and in Cells

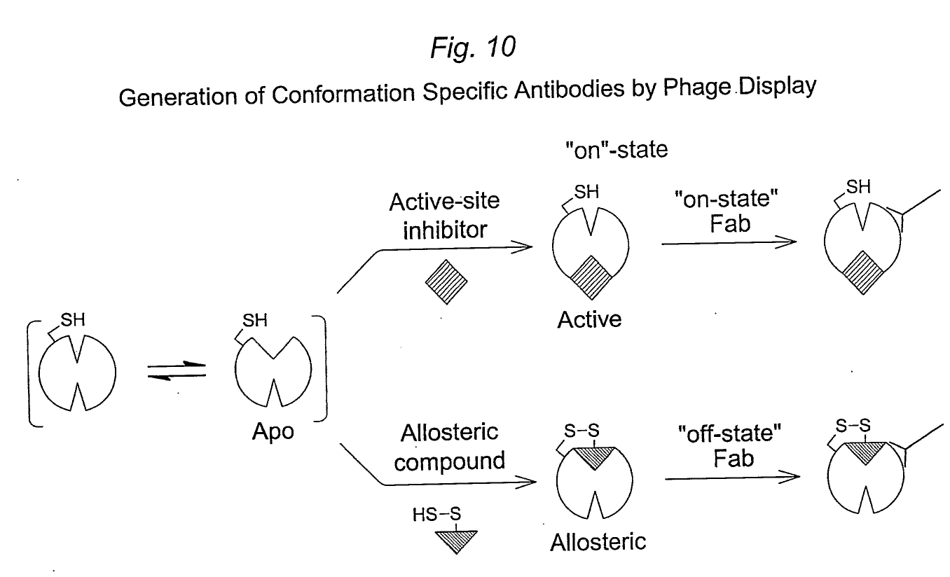

Given the large conformational change when the active site is occupied versus not, we proposed that it would be possible to generate conformationally selective antibody fragments (Fabs) to both the active state (“on-state”) and allosterically inhibited state (“off-state”) of the protein. It is known that antibodies can react to specific conformational states when obtained by classical immunizations and monoclonal antibody methods (for example see Jiang, J. et al., Mol Endocrinol, 18(12):2981-2996 (2004); Li, R. et al., Journal of Immunology, 168:1219-1225 (2002)). However, given the conformational dynamics of proteins and the uncertainties of protein antigen integrity when injected into a mouse, one cannot easily “trap” a single conformer and direct antibody production to the desired conformation. Using covalent probes directed to specific protein conformations, we show it is possible to trap mimics of the on- or off-state forms as homogeneous antigens to raise antibodies, specifically to human caspase-1 (FIG. 10). These antibodies are useful for trapping these forms both to probe their existence in cells and cell extracts, as well as, to drive conformational changes in the absence of added ligands. For example, one may want to activate an enzyme in a situation in which it is normally stored in an “off-state”. In this case one could add an “on-state” antibody and activate the enzyme. Similarly if one wished to turn off an enzyme one could add an “off-state” antibody. Since these antibodies are raised against epitopes away from the small molecules used to produce them, they will act in an allosteric fashion and thus not be prevented from binding based on direct physical exclusion by the small molecule ligands. In addition these conformationally selective antibodies can be useful for structural studies by stabilizing the protein. Thus, conformation selective antibodies will be very useful selective probes to detect, drive, and characterize the active or inactive forms of the enzymes in vitro and in cells.

Accordingly, we have produced Fabs by phage selection to the inhibited forms of the active and allosteric conformations of the enzyme. Phage display is a selection method based on direct affinity binding in vitro (see Lowman, H. B. and J. Wells, Journal of Molecular Biology, 234:564-578 (1993); Sidhu, S. et al., ChemBioChem, 4(1):14-25 (2002); Sidhu, S., Drug Discovery, ed. A. Carmen., Vol. 3. 2005, Boca Raton, Fla.: CRC Press. 748). Random protein or peptide variants are expressed on the surface of filamentous phage either in single or multicopy form and allowed to bind to the target protein immobilized on beads or plastic microtitre plates. Proteins that bind to the target adhere to the plates and are eluted after washing. The process is repeated 3-9 times to enrich for the best variants that bind to the target. The DNA sequence of the protein variant is readily cloned and sequenced since it is packaged in the filamentous phage or phagemid particle.

There are many advantages to using the phage display approach: it is fast and the fact that everything is done in E. coli greatly simplifies the expression of the final Fab fragment. Another major advantage is that the selections can be done in vitro so one can control the state of antigen throughout the selection process and importantly one can run counter-selections against forms of the antigen (including conformations) that one wishes to exclude (see Li, B. et al., Science, 270:1657-1660 (1995); Cunningham, B. and J. Wells, Current Opinion in Structural Biology, 7:457-462 (1997)). In this way we can enrich for Fabs for one state over the other. These experiments utilize codon-restricted synthetic libraries of antibody fragments on phage (see Sidhu, S., Drug Discovery, ed. A. Carmen., Vol. 3. 2005, Boca Raton, Fla.: CRC Press. 748). These libraries have been used extensively for selecting naïve antibodies for a multitude of targets (see Fellouse, F. et al., Journal of Molecular Biology, 357:100-114 (2006); Fellouse, F. et al., Journal of Molecular Biology, 348:1153-1162 (2005); Fellouse, F. et al., Proc Natl Acad Sci USA, 101:12467-12472 (2004)). Phage display is particularly useful for selecting conformationally selective antibodies. However, there are many possible alternative selection methods that would also work including ribosome display, yeast display, or any method where the “chemi-locked” conformation of the antigen is preserved throughout the selection process.

We used the Fab-phage to sort first for binding to the active or allosterically trapped forms of the caspase-1 labeled with either Ac-YVAD-CMK or compound #34, respectively. After two rounds of selection we counter-selected the Fab-phage by adding in solution the opposite-state form of caspase-1. Following four additional rounds of selection and counter-selection, individual clones that selectively recognized one conformation of caspases-1 over the other were detected and confirmed by spot phage ELISA. We prefer Fabs that can detect the protein conformation independent of the inhibitors. This way binding of the Fab will not require contact with the chemi-lock, and we can ensure the binding site is separate. To exclude Fabs that have the active site inhibitor as part of the binding epitope, we tested all the clones by spot ELISA for their binding to the active conformer trapped by a different active site inhibitor (z-WEHD-FMK). Similarly, a second allosteric compound (#11) was chosen to assess clones from allosteric screening. The results indicated that over two thirds of the clones were independent of the specific inhibitor structure. We selected those inhibitor independent Fabs for further study. We determined the affinities of these Fabs by competition phage ELISA. From the first selection, the affinities of the best off-state Fabs ranged from 300-600 nM and the best on-state Fabs showed an affinity of about 50-100 nM.

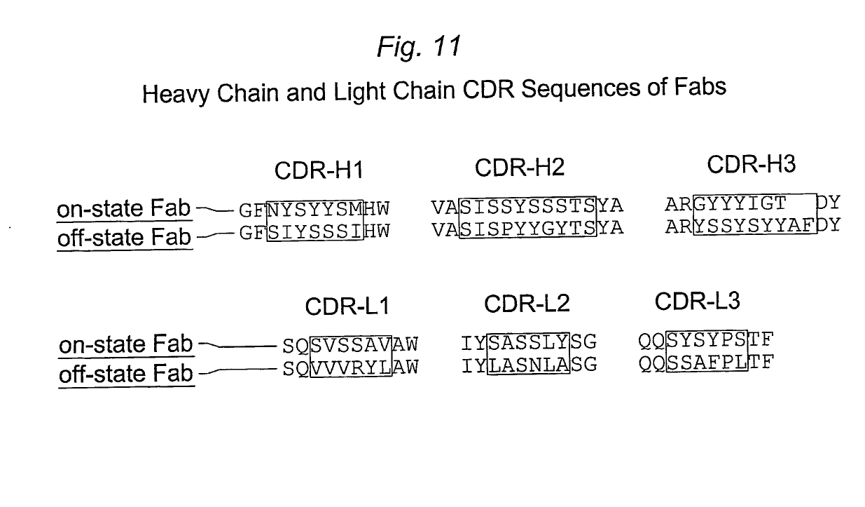

We have improved the affinity of on-state Fabs by partial randomization of all the three CDR loops on the heavy chain (see Fairbrother, W. et al., Biochemistry, 37(51):17754-17764 (1998)). We found changes in the CDR3 gave rise to the tightest binders and were about 20-fold improved in affinity (FIG. 11). A similar maturation strategy which involved partial randomization of light chain CDR loops has been applied to improve the affinity of off-state Fabs by 100-fold (FIG. 11).

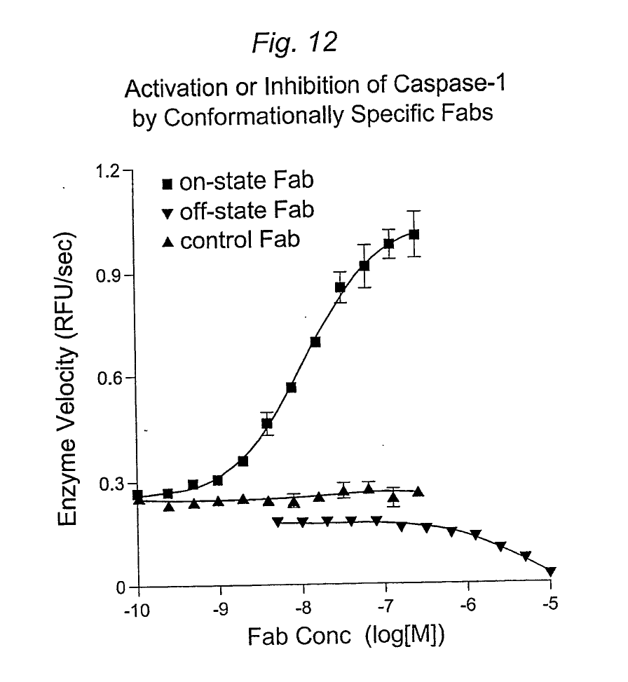

The tightest binding on-state Fab was expressed in E. coli and purified by protein A affinity chromatography in yields of ˜1 mg per liter culture media. Similarly, the yield of off-state Fab was ˜0.5 mg per liter culture media. Using a competitive ELISA, we found that both Fabs showed >15-fold selective for the opposite state of caspase-1. We tested the impact of the Fabs upon the activity of caspase-1. We found the on-state Fab enhanced the activity of the enzyme by about 3-fold compared to a non-cognate Fab made against VEGF. This is consistent with the on-state Fab driving the population of wild-type enzyme conformations into an on-state that is complementary to the on-state Fab. The measured EC₅₀ value (12 nM) is close to the binding affinity of on-state Fab to the on-state enzyme. Likewise, the off-state Fab inhibited the enzymatic activity with a Ki value of 0.92 uM (FIG. 12). These results are entirely consistent with the model that uninhibited caspase-1 is in dynamic equilibrium and that these Fabs can drive the enzyme into the corresponding state.

Thus, we have developed both small molecule traps (“chemi-traps”) and corresponding antibody traps (“immuo-traps”) for the active state and allosterically inhibited state. The Fabs will be important for biochemical and x-ray studies as well as cell biology experiments and as potential means of controlling caspase-1 activity.

### Example 7

### Sequence Comparisons of the Allosteric Sites of Caspase-1, -4, and -5 and Selectivity for a Disulfide-Trapped Compound

The sequence alignments of the large and small subunits in the putative allosteric regions of caspase-1, -4 and -5 display significant differences that may be exploited to develop enzyme specific probes (FIG. 13A). Of the 42 residues exposed in the allosteric cavity of caspase-1 (blue), 28 are conserved between caspase-1 and caspase-4 and -5. Of the remaining 14 exposed residues, two are conserved between caspase-4 and caspase-5 (colored red) and 4 are strictly unique to caspase-5 (green). An identity matrix shown of the entire large and small subunit regions of the inflammatory caspases indicates that caspase-1 is more distantly related to caspase-4 and -5 (59.4 and 56.8% respectively) and that the latter share 74.5% identity between themselves (FIG. 13B).

Compound #11 shows excellent selectivity for caspase-1 over both the highly conserved caspase-4 and caspase-5 and the less conserved executioner caspase-7. This compound specifically labels Cys331 in caspase-1 (FIG. 13C). Caspases-1, -4, -5 and -7 were treated with 100 uM compound #11 for 1 hr and then assayed for enzymatic activity (FIG. 13D). Whereas caspase-1 lost >95% activity, caspase-4 and -5 retained >90% activity. The sequence analysis and selectivity of labeling and enzyme inhibition indicates that ligands selective for each of the three inflammatory caspases can be developed using the methods of the present invention.

### Example 8

### Cellular Activity for Compound 11 Capped with Thiol-Ethyl Imidazole (IM-11)

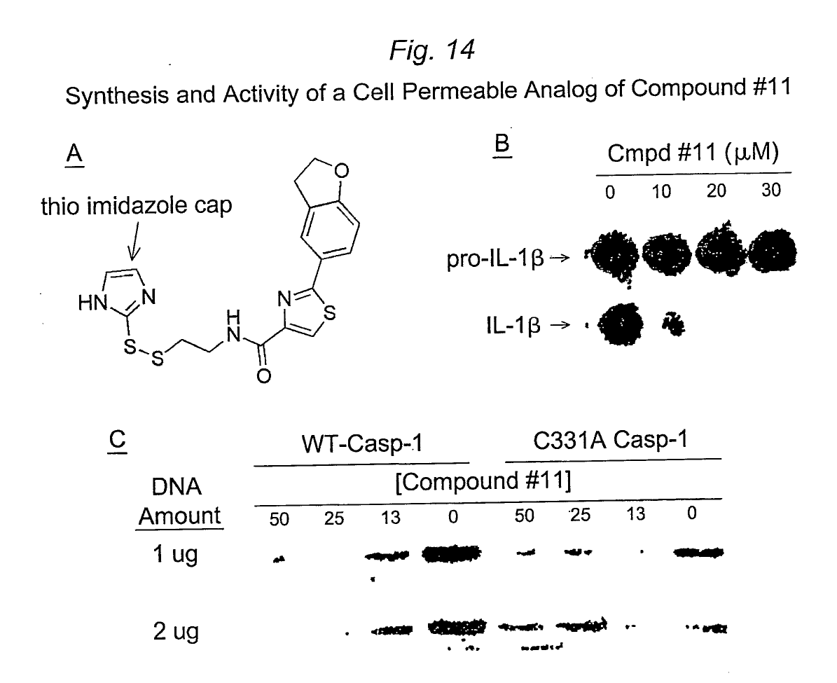

We have synthesized potentially cell permeable analogs of the six compounds described in Table 1 by replacing the cystamine cap with a neutral thiol-ethyl imidazole (IM) cap (FIG. 14A). These compounds were tested for inhibition of lipopolysaccharide induced IL-1β secretion from THP-1 cells, a human monocyte cell line frequently used as a model system for studying cytokine processing and secretion. Briefly, THP-1 cells were first treated with 1 mg/mL LPS for four hr to fully induce accumulation of pro-IL-1β (see Schumann, R. R. et al., Blood, 91(2):577-84 (1998)). Under these conditions, levels of processed IL-1β (17 kD)) are undetectable in cellular extracts, but high concentrations of pro-IL-1β are observed as expected. Processing of pro-IL-1β can then be rapidly stimulated by the addition of 5 mM ATP to the cells. Before addition of ATP however, the cells were treated with varying concentrations of IM-compounds for 25 min. The compounds (IM-3, -4, -11, -20, -32 and -34) were incubated with cells at varying concentrations up to 100 μM for one hr followed by activation of IL-1β production by treatment with 1 mg/mL LPS for four hr. Pretreatment with compound was followed by addition of 5 mM ATP and incubated at 37° C. for 15 min. Cells were collected by centrifugation and cellular extracts were made using M-PER mammalian protein extraction reagent (Pierce, Rockford, Ill.) and assayed by Western blot analysis for pro-IL-1β (31 kD) and processed IL-1β (17 kD). The results indicated that compound IM-11 blocked LPS induced processing and secretion of IL-1β and that the compound passed through the membrane and inhibited caspase-1 (FIG. 14B). The short duration of these experiments and presence of pro-IL-1β suggest that compound IM-11 is acting directly on IL-1β processing by inhibiting caspase-1 and not through a mechanism that decreases IL-1β transcription. We determined an average IC₅₀ value from four separate experiments of ˜5 μM for inhibiting LPS stimulated IL-1β for compound IM-11.

One of the advantages of disulfide-trapping is that by mutating the cysteine in the protein one reduces the potency of the disulfide compound by -50-100 fold (see Erlanson, D. et al., Proc. Natl. Acad. Sci. USA, 97(17):9367-9372 (2000)). This is because the thiol-disulfide exchange equilibrium contributes to the affinity as well as the non-covalent interaction between the compound and the target. This permits a simple specificity control. For determining the role of Cys331 in sensitivity to compound #11 in the THP-1 cell-based assay, we have used a pro-caspase-1 construct containing an alanine substitution at the Cys331 position to transfect THP-1 cells. These cells were compared to cells transfected with the wild-type pro-caspase-1 (FIG. 14C). The wild-type displayed a similar pattern of inhibition as the non-transfected cells, whereas the Cys331 mutant construct lost sensitivity to compound #11 in a DNA dose-depended manner. The decrease in total secreted IL-1β in the pro-caspase-1 C331A zero compound control lane is a reflection of the decreased catalytic activity of the mutant enzyme, as observed in kinetic analysis of the recombinant enzyme. We have produced a series of additional mutations at this site and measured the activity of the recombinant enzymes. This C331S mutant is only 2-fold reduced in catalytic activity over the WT enzyme. In summary, these data indicate that the mechanism of action of compound #11 inhibition of pro-IL-1β processing is through allosteric inhibition of caspase-1.

### Example 9

### Quantitation of Cell Permeability Mass Spectrometry

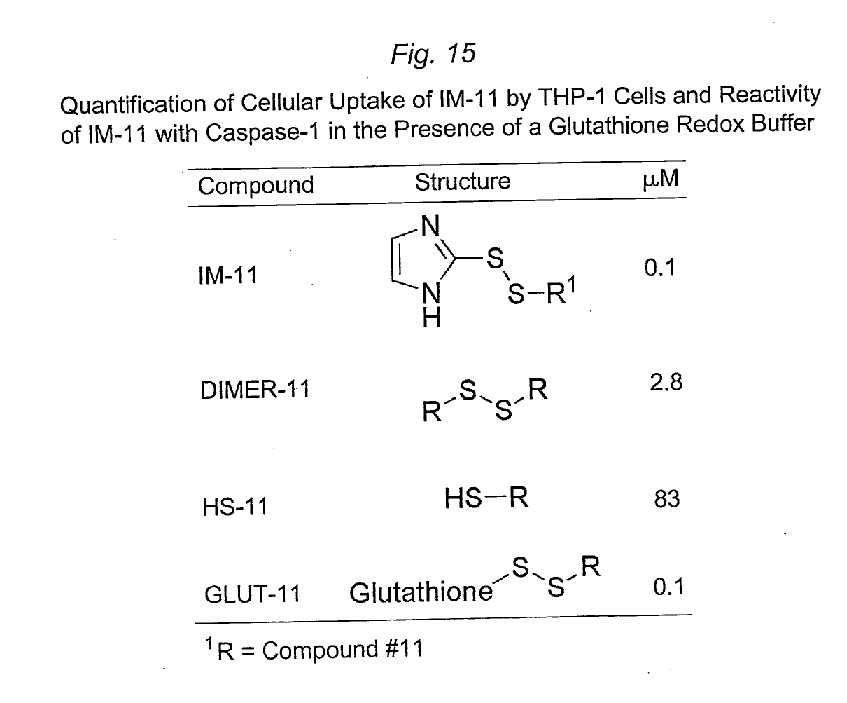

The data above indicated that compound IM-11 is cell permeable. To provide direct evidence, intracellular levels of the small molecule were measured by mass spectrometry as follows. Cells were incubated with 20 μM compound IM-11 for 30 min at 37° C. and subsequently washed three times with ice cold PBS. The washed cells were sonicated in 400 μL PBS and the debris and membranes were removed by centrifugation at 16,000 g for 30 min at 4° C. Both the cell extracts and the media were analyzed for quantification of compound IM-11 or other relevant derivatives (FIG. 15). To aid in this, we produced synthetic standards that represent the possible adducts in the cellular redox environment.

Compound IM-11 was observed to be mostly in the reduced form (HS-11) as would be expected in the reducing environment of the cytosol (FIG. 15). Thus, the compound was able to penetrate the cell and accumulated primarily in the reduced form to levels of ˜80 μM. The IC₅₀ in cells (˜5 μM) is very close to the DR₅₀ (˜10 μM) observed in the in vitro assay (Table 1) showing a good correlation between caspase-1 inhibitory activity and cell activity. We have also confirmed from in vitro experiments (not shown) that GSH/GSSG can promote thiol-disulfide exchange labeling and inactivation of caspase-1 in vitro.

### Example 10

### Caspase-1 High-Throughput Screen (HTS)

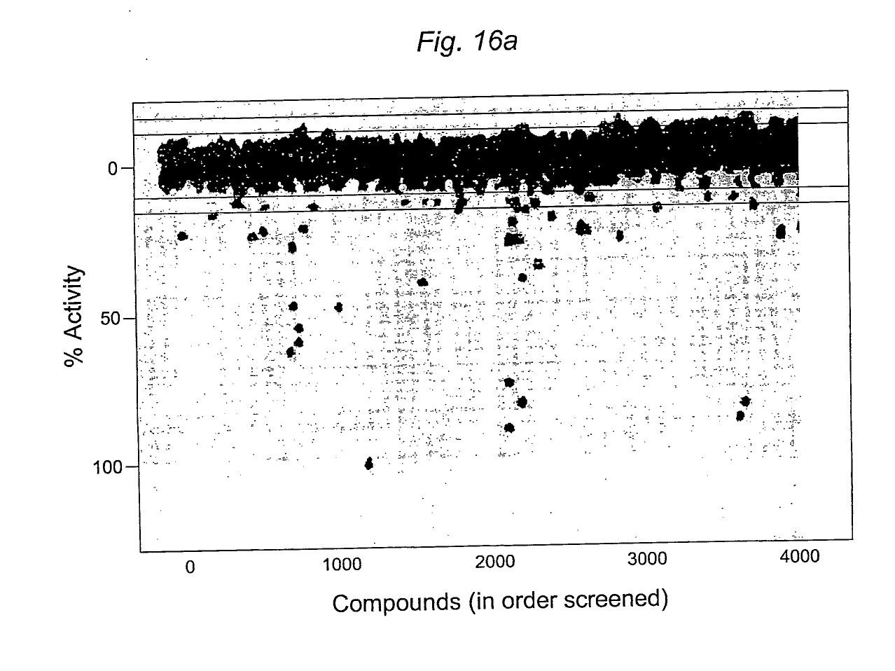

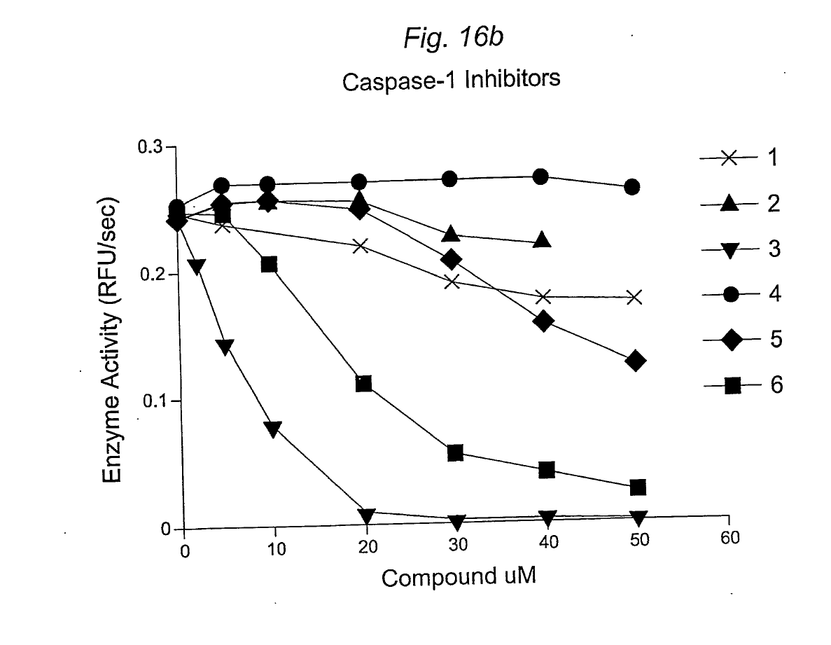

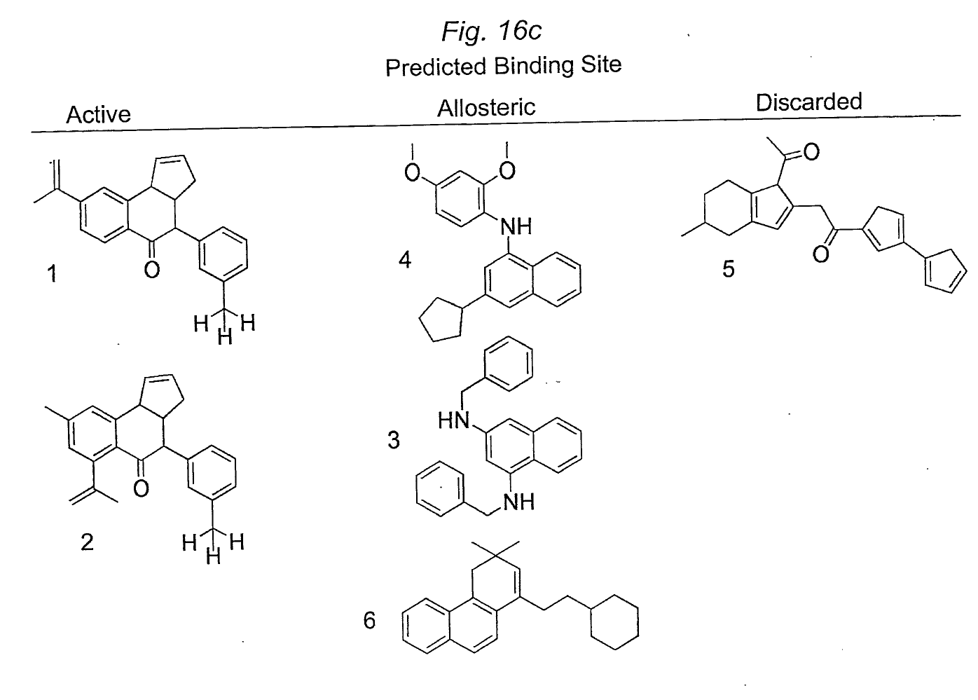

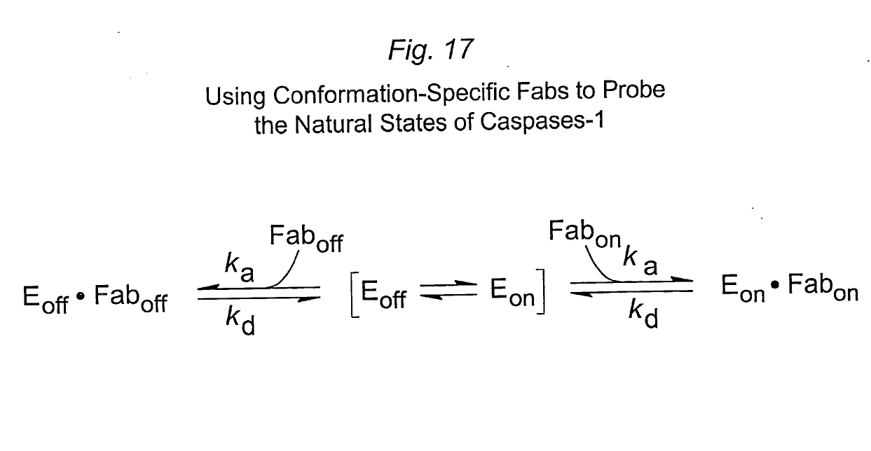

To identify new and free-standing compounds to the allosteric site we have recently screened caspase-1 against 10,000 compounds at the Small Molecule Discovery Center at UCSF. From this screen, 59 compounds were identified as hits with greater than 50% inhibition at 30 μM. The assay had a z-prime of 0.8661 and had a hit rate of about 0.6%. A scatter plot of the 10,000 compounds is shown in FIG. 16A. The hit rate and distribution for this screen validates this screening method to find modulators of caspase-1.

The IC₅₀ values representative compounds were measured and shown in FIG. 16B. Three classes of compounds were identified. One class contained carboxylate functionalities and may be active site inhibitors (FIG. 16C). A number of neutral compounds were identified that may be allosteric inhibitors. Some of these had IC₅₀ values 5 μM. Discernable structure activity relationship were observed from this set of compounds. These compounds do not inhibit caspase-7.

The published data (see Scheer, J. M. et al., Proc Natl Acad Sci USA, 103(20):7595-7600 (2006); Hardy, J. et al., Proc Natl Acad Sci USA, 101(34):12461-12466 (2004)) strongly support that caspase-1 can be allosterically regulated by small molecules captured by disulfide-trapping from a site at the dimer interface. We hypothesize that this allosteric mechanism is conserved within the other inflammatory caspases and may be used to naturally regulate these enzymes. Further experiments will analyze the circuitry of residues that connect the two active sites. Moreover, given the significant sequence variation at this allosteric site, and the fact that simple disulfide-trapping screens identified a highly selective compound for caspase-1 suggests it will be possible to generate selective compounds for the other inflammatory caspases. These inhibitors can be used to tease apart the roles of the different inflammatory caspases.

### Example 11

### Characterization of the Allosteric Circuit and Positive Cooperativity of Caspases using Mutational Analysis, Covalent Small Molecules, and Antibody Fragments to On- and Off-States of the Enzymes

We propose that the disulfide-trapped compounds disrupt a critical hydrogen bonding network in caspase-1 (an allosteric “circuit”). This network propagates allosteric interactions from one active site to the central cavity and through to the second active site. The function of this circuit can be tested using a systematic set of mutational and kinetic experiments. Our data from alanine-scanning mutagenesis for a number of these residues show dramatic effects on catalytic efficiency (Table 2). X-ray structures show these mutations do not misfold the enzyme (Scheer, J. M. et al., Proc Natl Acad Sci USA, 103(20):7595-7600 (2006) and our data). We further propose this circuit is critical for supporting positive cooperativity between the active sites, a surprising and recent discovery that is unique among proteases. It is plausible that the positive cooperativity may provide an additional selectivity filter for cleaving pro-inflammatory substrates known to be concentrated in cells. By testing the substrate concentration dependence for cleaving the pro-IL-1β, we can determine if this factor also stimulates positive cooperativity. We have shown that allosteric compounds discovered by disulfide trapping to caspase-1 and caspase-7 induce specific and natural structural transitions that mimic the apo- or zymogen-like conformation as seen by x-ray crystallography (see Scheer, J. M. et al., Proc Natl Acad Sci USA, 103(20):7595-7600 (2006); Hardy, J. et al., Proc Natl Acad Sci USA, 101(34):12461-12466 (2004)). The methods of this invention can be used to trap these different states using covalent active site and allosteric probes, and conformation specific antibodies. These tools can be used to validate and characterize these functional states both in vitro and in cell extracts.

### Example 12

### Mutational Analysis of the Allosteric Circuit and its Relationship to Positive Cooperativity in Caspase-1

How does binding at the allosteric site propagate to the active sites? We hypothesize these changes are mediated via a network of H-bonding interactions and side chains that allow or restrict key loop movements that form the active site. Furthermore, we suggest that the residues that propagate structural changes between the active and allosteric site also propagate changes between the active sites. We can test these hypotheses using alanine-scanning mutagenesis, enzyme kinetics, and x-ray crystallography on select mutants. The goal is to create a comprehensive mutational map that identifies side chains most responsible for propagating the conformational change between sites and stabilizes the active form of caspase-1.

The H-Bonding Network:

The discovery that disulfide-trapped compounds bind and disrupt a H-bonding network that radiates from the central cavity to the active sites suggests that mutations in this network could also disrupt the function of caspase-1. Thus, we produced a systematic set of mutations that truncated these side chains to alanine and measured their effects on k_{cat}, K_{M} and catalytic efficiency (k_{cat}/K_{M}) (Table 2). Our data shows dramatic functional effects for many of these mutations ranging from 20 to 3000-fold reductions in catalytic efficiency. These effects are as large as those seen when mutations are made in H-bonding groups that are known to directly stabilize the oxyanion transition state in serine and cysteine proteases (see Menard, R. et al., Biochemistry, 30:8924-8928 (1991); Braxton, S, and J. Wells, J. Biol. Chem., 266:11797-11800 (1991)). We recently discovered that caspase-1 shows positive cooperativity not seen in other proteases to date (see Scheer, J. M. et al., Proc Natl Acad Sci USA, 103(20):7595-7600 (2006)). Our data shows that most of the severely inactivating mutations also reduce the Hill-coefficient suggesting that we have uncoupled the positive cooperativity. There are several additional residues that can be tested, including Ser 339.

Hinge Points:

In addition to the H-bonding network that is disrupted upon binding of the disulfide-trapped compounds there are large conformational changes that occur between the off-forms of caspase-1 (disulfide-trapped or apo form) and the on-form (active site occupied). Gly-287 appears to play a major role in allowing this transition since it changes a net of 232° and 270° in φ and ψ angles, respectively, allowing the loop containing the active site Cys 285 and Arg 286 to twist in and out. Our data shows that the G287A mutation caused a massive 3100-fold reduction in catalytic efficiency. We propose this is due to alanine locking the enzyme off because the alanine side chain is not compatible with the φ and ψ angles seen in the active form of caspase-1. Confirmation of this can be obtained from solution of the x-ray structure of the G287A mutant. We have solved more than a dozen mutants and inhibited analogs of caspase-1 using well established procedures (see Scheer, J. M. et al., Proc Natl Acad Sci USA, 103(20):7595-7600 (2006); Romanowski, M. J. et al., Structure (Camb), 12(8):1361-71 (2004)). Our data shows that the P343A mutant enhances catalytic efficiency about 3-5 fold and slightly increases the Hill-coefficient. These data suggest Pro-343 may restrict the transition between on- and off-states. Further testing of this hypothesis can be performed using state-specific Fabs as described in Example 13 to see how the P343A mutant effects association rates (see below). Other residues that may also be important in conformational changes include Pro-290, located on the loop containing the active site cysteine (Cys285); Pro-387, located on the loop containing Glu390; and Gly346, which is located on the loop containing Arg341. The φ and ψ angles of this glycine are allowed for alanine in the active-site occupied crystal structure. However, in the allosteric conformation, the dihedral angles of this glycine are conformationally inaccessible to alanine, suggesting that the G346A mutant may adopt an “active-state locked” conformation, the opposite of the G287A mutant.

### Example 13

### Production of Fabs to Characterize the “on” and “off” States of Caspase-1

We have proposed (see Scheer, J. M. et al., Proc Natl Acad Sci USA, 103(20):7595-7600 (2006)) that the free processed form of caspase-1 exists in dynamic equilibrium between two basic states, an on-state and off-state (FIG. 10). These two states may over-simplify the actual situation, since there may be an ensemble of states within the on-state and off-state populations, and we may not have sampled them with our two classes of covalent labels. Nonetheless, the two states we observe crystallographically provide a useful working model. We hypothesized that if caspase exists in these two states that are trapped by the covalent inhibitors (chemi-locks), then it may be possible to capture these two conformations with Fabs. Comparison of the surfaces of the two inhibited forms does show significant differences especially near the dimer interface and the active sites. These Fabs would be very useful functional probes for these conformations in wild-type, mutants and small molecule inhibited forms of caspase-1.

Our results show that we have succeeded in producing potent Fabs with low nM affinity to both the active site inhibited (on-state) and allosteric site inhibited (off-state) forms of caspase-1 that show >15-fold affinity difference against the opposite locked forms (FIG. 11). Both the on-state and off-state Fabs have been expressed in E. coli and purified by protein A affinity chromatography. We have characterized the binding constants, association rates (kₐ) and dissociation rates (k_{d}) for the on-state, off-state and apo forms of caspase-1 (non-inhibited wild-type) using BIAcore measurements. We immobilized each form of caspase-1 to the chip and flowed increasing concentrations of variants of Fab (Table 3). As predicted, the on-state Fab bound over 100-fold weaker to apo caspase-1 than to the active-site inhibited form, and the off-state Fab bound to the uninhibited form with an affinity only three-fold less than to the allosterically inhibited enzyme. Furthermore, virtually all the observed affinity differences showed up in the association rates (kₒₙ) not dissociation rates (k_{off}) for the Fabs. These data strongly suggest the existence of two conformational populations of caspase-1 in solution and the uninhibited caspase-1 closely resembles the allosterically inhibited form.

TABLE 3
Kinetic analysis of Fabs against different states of caspase-1 by BIAcore

| | on-state caspase-1 | | | off-state caspase-1 | | | apo caspase-1 | | |
|---|---|---|---|---|---|---|---|---|---|
| | K_{D} | kₒₙ | k_{off} | K_{D} | kₒₙ | k_{off} | K_{D} | kₒₙ | k_{off} |
| | (10⁻⁹M) | (10⁴ s⁻¹) | (10⁻³ M⁻¹s⁻¹) | (10⁻⁹M) | (10⁴ s⁻¹) | (M⁻¹s⁻¹) | (10⁻⁹M) | (10⁴ s⁻¹) | (10⁻³ M⁻¹s⁻¹) |
| on-state Fab | 2.5 | 66 | 1.7 | N.D. | N.D. | N.D. | 330 | 0.8 | 2.6 |
| off-state Fab | 99 | 1.6 | 1.6 | 4.7 | 135 | 6.4 | 17 | 55 | 9.5 |

These Fabs are useful for corroborating and interpreting mutational data from above. For example, mutations in the allosteric circuit that destabilize the on-state relative to the off-state (such as R286A, E390A or G287A) may show much reduced affinities toward the on-state Fab and little or slight affinity improvement for the off-state Fab. If the P343A mutation reduces the energy barrier between the off- and on-states, then this mutation may enhance binding to the on- or off-state Fabs depending on equilibrium point for caspase-1. Thus we will determine the kinetics and affinities for these Fabs and selected mutations.

Since both of the Fabs bind to the uninhibited enzyme, we will determine if they compete for the same binding epitope on caspase-1 by BIAcore analysis. This will provide low-resolution information about the relative positions of the binding epitopes. We can also perform X-ray crystallography of the complex between each Fab and caspase-1. We have produced ˜50 mg quantities of caspase-1 and each Fab. These were mixed in 1:2 ratio of caspase dimer:Fab and put through a typical crystal screen to identify crystallization conditions by hanging drop vapor diffusion methodology (see Hardy, J. et al., Proc Natl Acad Sci USA, 101(34):12461-12466 (2004); Romanowski, M. J. et al., Structure (Camb), 12(8):1361-71 (2004)). We have obtained crystals at reasonable size for the caspase-1/on-state Fab. This will yield high resolution information about where each Fab sits and allow us to compare the structures the Fabs trap to those the small molecule inhibitors trap.

### Example 14

### Characterization of Positive Cooperativity Using on-State Locked and Mutated Heterodimers of Caspase-1

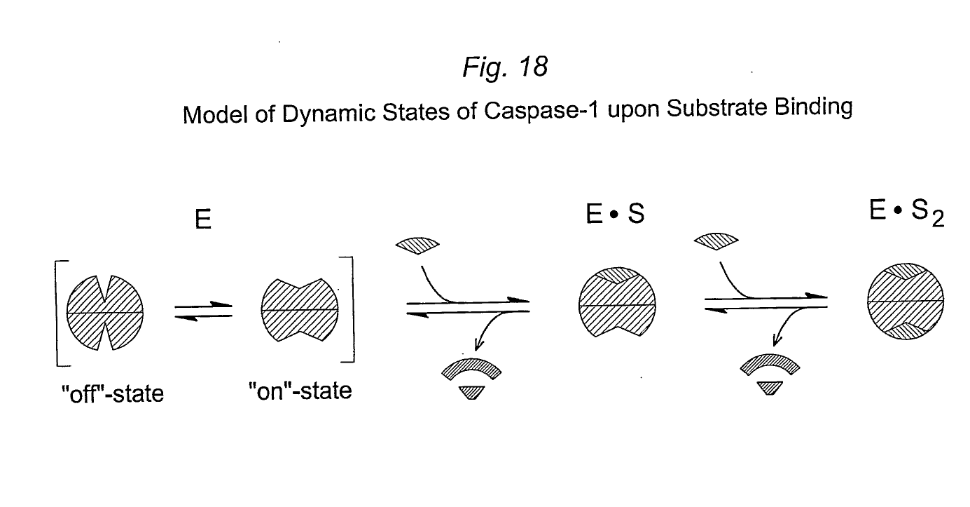

Positive cooperativity suggests that the enzyme is better at catalyzing turn-over when both sites are occupied than when one site is occupied. A simple model of four possible states is depicted in FIG. 18. The measured Hill-coefficient of 1.5 for wild-type caspase-1 suggests a good degree of coupling between the two catalytic sites that is comparable to phosphorylase A (see Buchbinder, J. L. et al., Biochemistry, 34(19):6423-32 (1995)). However this does not reveal the kinetic constants for binding or turnover of substrate from either the E•S or E•S₂ complex. Our data using different tagged and labeled large subunits show we can produce pure heterodimers where one site is occupied by a covalent substrate analog and the other site is free (FIG. 8). We suggest this produces a mimic of the E•S state. This allows us to determine what the catalytic enhancement (or inhibition) is when one active site is occupied. Remarkably, we found there is a two-fold enhancement in catalytic efficiency (k_{cat}/Kₘ) in the E•S locked heterodimer relative to the unmodified enzyme (Table 4). We will evaluate the Hill-coefficient for this enzyme which we would anticipate to be near 1.0. We will also test the binding of our on-state and off-state Fabs to the E•S locked heterodimer by BIAcore to compare the kinetics and binding affinity to their parent antigens and the unmodified caspase. If the conformation of the E•S is essentially like E•S₂ then we should see virtually the same affinity and kinetics for the E•S locked heterodimer (i.e. single active site labeled) and the E•S₂ locked homodimer (i.e. double active-site labeled). Lower affinity for the E•S locked heterodimer would suggest at least one subunit has an intermediate conformation between the on- and off-states. We will also test to see if the on-state Fab has the same affinity when BIAcore analysis is done in the presence of saturating substrate. Assuming the conformation of caspase-1 is the same whether it is bound by substrate or labeled by an active site inhibitor, then the binding constant between on-state Fab and the enzyme should be the same for the substrate saturated caspase-1 or active-site labeled caspase. Thus, we will add substrate at ten-fold above Km and measure association rates for the on- and off-state Fabs.

TABLE 4
Kinetic analysis of caspase-1 hybrid constructs

| | K_{M} | k_{cat} | k_{cat}/K_{M}, | Ratio |
|---|---|---|---|---|
| Construct | μM | sec⁻¹ | M⁻¹ · sec⁻¹ | k_{cat}/K_{M} |
| Unlabeled hybrid | 1.9 | 0.11 | 5.6 × 10⁴ | 1 |
| Half-labeled hybrid | 3.8 | 1.93 | 5.1 × 10⁵ | 9.1 |

The E•S locked heterodimer experiment suggests that the catalytic efficiency from the E on-state is at least 30-fold lower than from the E•S state (FIG. 18). The E•S locked heterodimer experiments above allow us to analyze the kinetics from the E•S state. However, it does not tell us what the kinetics are from the E on-state. To measure these, we will construct a heterodimer in which we have inactivated catalysis and binding by introducing the catalytic C285A and the P1 binding site R179A mutations. This heterodimer will have one fully functional active site and one that can neither bind nor catalyze hydrolysis so that it is only capable of going between E and E•S.

Our data shows the on-state Fab enhances catalysis of caspase-1 about 3-fold (FIG. 12). Moreover, this catalytic effect titrates with the IC₅₀ of the on-state Fab and is unaffected by titration with a non-binding Fab. We suggest this Fab stabilizes the on-state of E (FIG. 18) and essentially works to lower the transition barrier between the off and on-states much like the P343A gate-keeper mutation. We can test this by adding the on-state Fab to the P343A gate-keeper mutant. If the barrier between off- and on-states is fully lowered by either effect alone, then adding the on-state Fab to the P343A mutation would have no further catalytic enhancement.

The k_{cat} and K_{M} values for pro-IL-1β, the natural substrate of caspase-1 has not been reported for comparison with synthetic substrate hydrolysis. Moreover, it has been recently suggested that caspase-1 cleaves pro-IL-1β that has been concentrated at membranes or even in vesicles (see Ferrari, D. et al., Journal of Immunology, 176(7):3877-3883 (2006); Andrei, C. et al., Proc Natl Acad Sci USA, 101(26):9745-9750 (2004); MacKenzie, A. et al., Immunity, 15:825-835 (2001)). This is consistent with the positive cooperativity we observe with synthetic substrates. Thus, it is important to determine the kinetic constants and Hill-coefficient for hydrolysis of pro-IL-1β to see how these values track the positive cooperativity we observed with synthetic substrates. To do this, we will determine the rate of cleavage of recombinant pro-IL-1β as a function of its concentration by monitoring both the production of IL-10 and depletion of pro-IL-1R using a western blot assay we have developed (FIG. 14) (see Black, R. A. et al., J Biol Chem, 263(19):9437-42 (1988)). We can also follow the hydrolysis time course by mass spectrometry as necessary. These data will provide biochemical validation for positive cooperativity on a natural protein substrate, and important biochemical characterization for cell-based work that follows in Examples 16 and 21.

### Example 15

### Analysis of Cooperativity and Common Allosteric Features for Caspase-4 and -5

Caspases-4 and -5 are the closest homologs of caspase-1. Caspase-5 is known to play a role in processing of pro-inflammatory cytokines along with caspase-1, and caspase-4 may sit upsteam of caspase-1 (see Faucheu, C. et al., Eur J Biochem, 236(1):207-13 (1996)). These enzymes are about 55%-75% identical in residues that line the allosteric site, and many of the alanine mutants in the allosteric circuit that affect the functions of caspase-1 are conserved between caspases-4 and -5 (FIG. 13). Thus we hypothesize these same residues will serve a similar function in caspases-4 and -5 as seen in caspase-1 and both will show positive cooperativity. Recombinant forms of large and small subunits of caspase-4 and -5 have been expressed as inclusion bodies in E. coli and re-folded into the active enzymes (FIG. 5) using the procedure we have developed for caspase-1 (see Romanowski, M. J. et al., Structure (Camb), 12(8):1361-71 (2004)). We will determine if the enzymes show positive cooperativity by standard Michaelis-Menten analysis. Our mutational studies will be guided by homology models. Accordingly, high quality homology models for these enzymes will be developed. We will selectively mutate the allosteric site salt bridge residues (Arg286 and Glu390), the hinge residue (Gly287), and the S-1 site gate-keeper (at position 343) to test if these residues play similar roles. Interestingly, Pro343 is a serine and arginine in caspase-4 and -5, respectively (FIG. 13). We would predict that homology models of caspase-4 and -5 may show these potential H-bonding residues are positioned to restrict the s1 loop movement. If this is the case, the alanine mutations should disrupt these restrictive interactions an enhance activities of caspases-4 and -5 as seen for the P343A mutant in caspase-1.

These mutational and mechanistic studies will provide the foundation for the comparative enzymology of the inflammatory caspases. An integrated approach that employs mutational analysis, heterodimer construction, kinetics, structural analysis of trapped small molecules and antibody traps provides a much clearer picture of the allosteric circuitry and mechanistic basis for positive cooperativity. The tools and understanding developed at this stage in vitro can be employed for probing the cellular relevance and functions of caspases in vivo. These studies and reagents will be useful for characterizing other synthetic or natural inhibitors as described below. This general approach will be broadly relevant to studying allosteric transitions in other proteins as well.

### Example 16

### Identification and Characterization of Selective and Cell Active Allosteric Inhibitors to Caspase-1 and a Screen for New Synthetic and Natural Inhibitors

We can study the selectivity for cell-based inhibition of caspase-1 by the disulfide-trapped compounds. Our data show that when the allosteric thiol in caspase-1 is replaced in THP-1 cells, the compounds no longer inhibit processing of IL-1β (FIG. 14). We have worked out a scheme for synthesizing an [³⁵-S]-labeled allosteric compound that can be used to directly verify labeling of caspase-1 or other proteins in THP-1 cells. Our data has shown that the disulfide-trapped compounds readily exchange into caspase-1 in a GSH/GSSG redox buffer that mimics intracellular conditions. We will use these conditions as a useful secondary screen for additional cell active compounds. The skilled artisan will appreciate that the allosteric site represents a drug target site. We have developed a HTS assay for caspase-1, screened 10,000 compounds and have identified non-carboxylate containing hits. These hits can be tested using our conformationally trapped forms of the caspases to identify potential allosteric inhibitors. Also, we can search for natural inhibitors of caspase-1 that may exist to shut down caspase-1 following the transient burst of pro-inflammatory cytokine processing in THP-1 cells. We will purify and characterize this form of caspase-1 in THP-1 cells using our conformationally sensitive antibody probes.

### Example 17

### Characterization of the Selectivity for Disulfide-Trapped Compounds to Caspase-1 in Vitro Versus Caspase-4 and -5

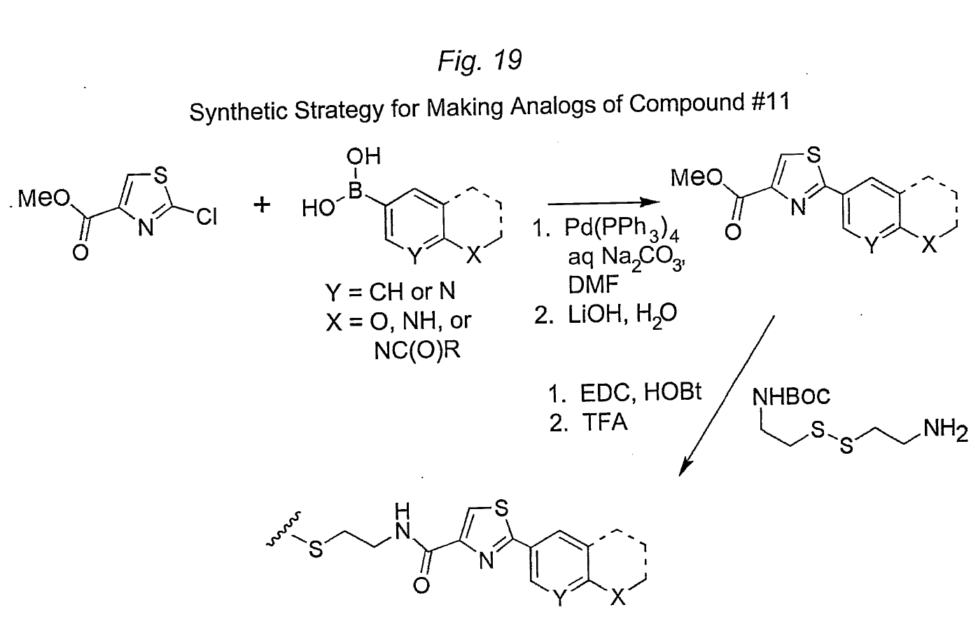

In order to dissect apart the specific roles of inflammatory caspases in cells relative to pro-IL-1β processing it is important to have small molecule inhibitors that are shown to be selective to the target in vitro. The allosteric sites of caspases-1, -4 and -5 show only 55-75% identity in the allosteric pocket and are much less conserved than their active sites (FIG. 13). Our data shows that we have an allosteric inhibitor (compound #11) that is between 25 to 100-fold selective over caspase-4 and -5 both with respect to the extent of conjugation (FIG. 13C) and level of inhibition of activity (FIG. 13D). This difference should be sufficient for the cell-based experiments described herein. However, if one needed to increase selectivity, the synthetic strategy for making analogs of compound #11 that can be tested for potency and selectivity relative to the other caspases is shown in FIG. 19. There are many commercially available boronic acids which can be coupled to the chloro-thiazole ring which can be used. A library of different analogs can be made and tested for conjugation strength and selectivity.

Compound #11 can be made virtually ineffective with caspase-1 by mutating the allosteric cysteine to alanine (C331A). For example, the C331A mutant of caspase-1 is not significantly inhibited at concentrations up to 50 μM by compound #34, whereas the wild-type caspase-1 has an IC₅₀ of ˜10 μM in 1 mM β-ME or in cells (Table 1 and FIG. 14B). Thus, to ensure greater selectivity between the compounds that react with caspase-1 versus the other inflammatory caspases we will mutate the allosteric thiols in caspases-4 and -5 and thus protect them from residual cross-reactivity from disulfide inhibitors to caspase-1. The allosteric thiol mutants are very useful controls and provide a unique advantage of disulfide trapped probes.

### Example 18

### Characterization of the Potency and Selectivity of Disulfide-Trapped Compounds for caspase-1 in THP-1 cells

Our data on compound #11 capped with an imidazole moiety shows good potency in blocking pro-IL-1β in THP-1 cells (IC₅₀˜5 μM; FIG. 14B). Furthermore, when THP-1 cells are transfected with caspase-1 containing a C331A mutation, compound #11 no longer blocks the processing of pro-IL-1β (FIG. 14c). Thus, in vitro and in cells the C331A variant of caspase-1 protects it from compound inhibition even to concentrations up to 50 μM. We will determine the IC₅₀ values for processing of other the pro-inflammatory proteins (i.e. pro-IL-18, pro-caspase-1, pro-caspase-5 and pro-caspase-4) using western blot assays to compare with the IC₅₀ value for inhibition of pro-IL-1β processing.

We have developed a synthetic scheme for producing the [³⁵-S]-cysteamine based on published methods (see Harapanhalli, R. S. et al., Nucl. Med. Biol., 20(1):117-124 (1993)). This will be attached to the corresponding acid precursor of compound #11 and others to be tested that contain the cysteamine linker. The [³⁵-S]-labeled compound #11 should provide direct evidence that the compound is selectively labeling caspase-1 in cells. Immunological pull-downs using antibodies specific for the caspases can be used isolate each enzyme, followed by non-reducing SDS-PAGE and autoradiography. Cells will be treated with the [³⁵-S] labeled compound and proteins from cell extracts will be prepared by reacting all free thiols with excess iodo-acetamide to prevent thiol scrambling. Proteins will be electrophoresed on non-reducing one dimensional gels to detect labeling of caspase-1 and to determine the extent of peripheral labeling. At a specific activity of 1mCi/mg, we calculate that one should be able to detect as little as 5% of total caspase-1. One can avoid extensive peripheral labeling because the closest homologs in the genome to caspase-1 are caspase-4 and -5, and if only highly selective caspase compounds are used. Even if labeling occurs to peripheral sites, it is likely not to be relevant to rapid caspase signaling events we are monitoring. Recent work has shown highly selective thiol labeling to Rsk kinase with a compound containing a fluoromethyl-ketone covalent warhead attached to a fairly weak non-covalent binding entity (see Cohen, M. et al., Science, 308:1318-1321 (2005)).

If significant non-selective labeling is observed with compound #11, we will test other analogs or screen for other compounds as out-lined in Example 17. These compounds should readily penetrate cells since they are neutral and small (MW 250-300) and the charged cysteamine cap is easily replaced with a neutral thiol-ethyl imidazole moiety (see FIG. 14) or others to facilitate their transport. In the event that neutral caps do not generally facilitate transport we can use the TAT peptide disulfide system that has been shown to be an effective means of facilitating delivery of peptides and proteins into cells as a disulfide conjugate to the TAT peptide (see Schwarze, S, and S. Dowdy, Trends Pharmacol. Sci, 21:45-48 (2000); Hallbrink, M. et al., Biochimica et Biophysica Acta, 1515:101-109 (2001)). The intracellular (GSH/GSSG) redox buffer is close to the redox buffer we employ in our in vitro screen (β-ME-SH/β-ME-S-S-ME-β). Our data show that we observed effective and comparable disulfide-trapping of compound #11 in vitro using a GSH/GSSG redox buffer that brackets the anticipated cellular conditions (data not shown). These cellular experiments will serve as the model approach for analyzing other disulfide-trapped compounds both for the inflammatory caspases as well as for any other cellular targets.

### Example 19

### Characterization of Hits from High Through-Put Screening of Caspase-1

We have developed a robust HTS assay for inhibitors of caspase-1. We have completed a screen of 10,000 drug-like small molecules and identified 59 compounds that caused >50% inhibition of caspase-1 at 30 μM (FIG. 16). Several of these compounds (#3, 4 and 6; FIG. 16) lack the carboxylate group that is characteristic of compounds that bind to the active site. These may be allosteric inhibitors. The diaminoquinazoline compound (compound #3, FIG. 16) is particularly interesting with an IC₅₀ of 5 μM. Additional screening of >70,000 compounds for inhibitors of caspase-1 at the Molecular Libraries Screening Center Network through NIH can be performed. These additional hits for caspase-1, as well as previous ones, will be triaged as described below.

IC₅₀ values will be determined in 0.1% Triton X-100 to eliminate promiscuous aggregating molecules (see Feng, B. et al., Nature Chemical Biology, 1(3):146-148 (2005)). We will determine the solubilities of the more potent compounds to further ensure we are not observing precipitation effects. We can test the most potent and soluble compounds against caspase-4 and -5 to determine their selectivities. We will determine the mechanism for inhibition by Michaelis-Menton analysis. We would expect that specific inhibitors to either the active site or allosteric site would show competitive inhibition since binding to either is mutually exclusive. However, non-specific inhibitors would show non-competitive inhibition and would be eliminated.

We will use the assays developed in Example 11 for distinguishing the different sites these compounds bind. For example, we would expect that compounds that block the active site would inhibit the E•S locked heterodimer described in 1c, whereas allosteric site compounds would neither bind nor inhibit. Compounds that bind to the allosteric site should stabilize the off-form of the protein and thereby reduce binding affinity for the on-state Fab. Such compounds may improve binding for the off-state Fab. Thus, we will analyze the Fab IC₅₀'s by ELISA. These simple assays will allow us to distinguish active site and allosteric inhibitors. Compounds that pass these tests will be used to determine X-ray structures to confirm the binding mode. We will also determine the IC₅₀ values to inhibit caspase-1 in THP-1 cells.

Compounds identified from screening allow a means to get to soluble compounds that are selective and specific for caspase-1. Such compounds can be used as starting points for medicinal chemistry to advance to lead optimization. Simple analogs could be purchased first to develop a simple SAR. These can be followed up with more detailed chemical synthesis depending on the nature of the compound.

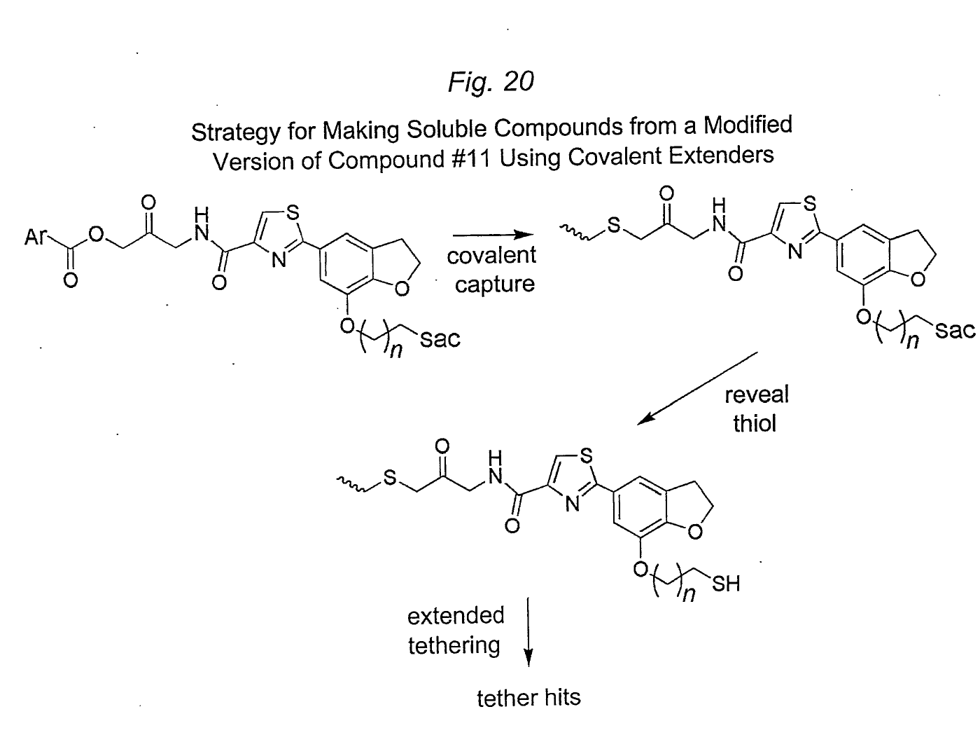

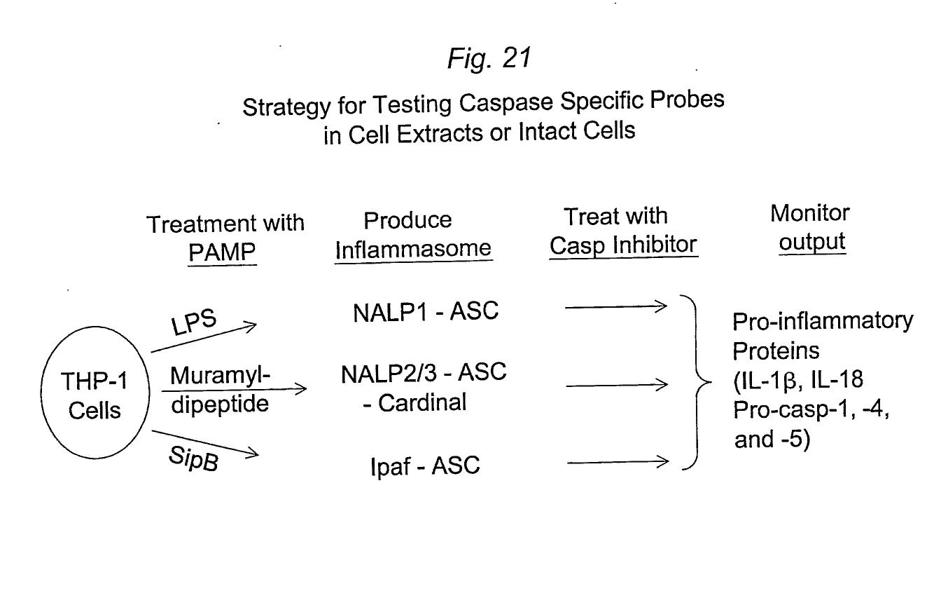

As needed, it is also possible to generate soluble compounds from the disulfide-trapped fragments using covalent extenders (see Erlanson, D. et al., Nature Biotechnol., 21(March):308-314 (2003)) as out-lined for compound #11 in FIG. 20. A derivative of compound #11 will be synthesized which can alkylate the allosteric cysteine. The protein will be alkylated with the compound and treated at pH 9 for 1 hr to hydrolyze the thiol-acetyl and reveal the free thiol. The derivative will be used for disulfide screening to identify new hits that conjugate to the exposed thiol on the thiazole-dihydrobenzofuran fragment. Soluble analogs can be made by producing the terminal amide on the thiazole ring and surrogating the disulfide to the new fragment with an ethylene bridge. Soluble compounds can then be assayed as above.

### Example 20

### Probe for a Natural Inhibitor of Caspase-1

The mechanism for turning off caspase-1 in the cell is unknown. Unlike caspase-7 which can be inhibited by XIAP, there are no examples of natural inhibitors of caspase-1 yet published. There are several lines of investigation that can be used to probe for a natural regulator for activated caspase-1. It is known that caspase-1 is secreted from the cell along with IL-1β after processing (see Ferrari, D. et al., Journal of Immunology, 176(7):3877-3883 (2006); Andrei, C. et al., Proc Natl Acad Sci USA, 101(26):9745-9750 (2004); MacKenzie, A. et al., Immunity, 15:825-835 (2001)). It is possible there are natural inhibitors of caspase-1 in serum. We will add caspase-1 to serum and determine if we observe inhibition. If we observe inhibition it will be useful to see if it inhibits both caspase-4 and -5. It would be useful to see if it inhibits the E•S locked form of the caspase-1 and affects binding of the on-state and off-state Fabs to determine which state it may be interacting with. If we observe inhibition and identify which state, we can use our on-state and off-state Fabs to pull down a potential protein complex which could be observed by SDS-PAGE and identified by mass spectrometry. If it appears to be a small molecule, we would dialyze and fractionate serum to identify inhibitory fractions for further characterization.

It is also possible that an inhibitor is produced in THP-1 cells and combined with caspase-1 after processing. We will stimulate THP-1 cells with LPS and ATP and pull down the endogenous caspase-1 using the on-state and off-state Fabs and analyze for a bound protein as above. One obvious candidate would be proteolytic fragments from processing of pro-IL-1β. These may even be seen in the in vitro kinetic experiments proposed in Example 14. Yet another possibility is that caspase-1 is inactivated by reactive oxygen species at the allosteric cysteine-331, or active site cysteine-285. In fact, several groups have suggested nitric oxide may regulate caspase-3 by thiol nitrosylation (see Matsumoto, A. et al., Science, 301:657-661 (2003); Mitchell, D. and M. Marietta, Nature Chemical Biology, 1(3):154-158 (2005)). A covalent modification can be probed by isolating caspase-1 that is secreted from THP-1 using the on-state and off-state Fabs and characterizing the protein by mass spectrometry. We will dock metabolites into caspase-1 that may bind to either the active or allosteric sites of caspase-1 using a robust docking algorithm for metabolites (see Kalyanaraman, C. et al., Biochemisty, 44:2059-2071 (2005)). The best candidates can be tested in vitro to determine if we see inhibition at physiologically relevant concentrations.

Finding a natural regulator would greatly advance our understanding of the biology of the inflammatory caspases just as the XIAP's were a break-through for understanding the regulation of the apoptotic caspases.

### Example 21

### Determination of the Roles of Caspases in Driving Innate Cellular Immune Responses

In Example 21, whether the allosteric regulatory site in caspase-1 is present in other inflammatory caspases resident in THP-1 cells can be determined. Thus, we will screen the disulfide compound library for inhibitors. Our caspase-1 compounds are highly selective for caspase-1. Thus, the skilled artisan will appreciate that it will be possible to generate highly selective compounds to other inflammatory caspases. Allosteric inhibitors can have advantages over active site inhibitors in two ways. First, the inflammatory caspases have virtually identical substrate specificities (see Thornberry, N. A., Br. Med. Bull., 53(3):478-90 (1997)) making the active site more challenging for obtaining specific compounds. The central cavity is less conserved than the substrate binding groove and thus has greater potential for identifying specific compounds. Second, the active sites of these enzymes have so far been intractable to generating good drug leads owing to a strict requirement for an electophillic warhead linked to an aspartyl functionality. We will use the allosteric inhibitors to trap and characterize the allosteric transitions within each protein and employ them as selective probes to determine the roles of these enzymes in promoting the cellular inflammation response.

### Example 22

### Screening for Caspase-4 and -5 Inhibitors and Selectivity Tests In Vitro

To simplify the screening for disulfide-trapped compounds we will mutate non-allosteric and exposed cysteine residues to alanine in the small subunit of caspase-4 and -5, as we did for caspase-1, and determine their effects on enzyme activity. It is not anticipated that removing these surface thiols will be structurally disruptive as most are in the same positions as in caspase-1. Caspase-4 has one additional thiol (Cys363) and caspase-5 has two additional thiols (Cys319 and Cys363) relative to caspase-1. If we do have a problem replacing these surface thiols and retaining wild-type activity, we will perform the screen with them intact and subsequently deconvolute labeling within the small subunit by comparing the labeling patterns for the small subunit with and without the allosteric cysteine.

Given our success in identifying allosteric inhibitors for caspase-1, -3 and -7, we fully expect that we will be able to find disulfide-trapped compounds that inhibit caspase-4 and -5. Each enzyme will be screened with a ˜10,000 member thiol-fragment library to identify primary hits. Hits will be triaged in a manner similar to that for caspase-1 except we will focus on compounds that show the greatest structural diversity from hits seen in the other two caspases to better ensure selectivity. Hits from the primary screen will be confirmed by resynthesis. Conjugation strength will be determined by DR₅₀ and β-ME₅₀ measurements on the wild-type caspase-4 and -5. We will measure the relationship between the extent of enzyme inhibition versus the labeling to establish the stoichiometry of labeling that correlates with full inhibition. To determine that the functional effects are driven by disulfide formation at the allosteric site, we will evaluate if the effects are fully reversible by reduction. In the event that we do not find inhibitors by screening the allosteric thiol, we can introduce and screen additional thiols around the cavity. Such thiols can be easily designed from homology models of caspases-4 and -5. As long as our designed thiols are within 5-7 Å of the binding site it is very likely we will find hits given the breadth of the disulfide library and the intrinsic flexibility of the thiol linkers. Such thiol mutants can still be employed in extract or cellular studies since we can add the mutant enzymes exogenously or by transfection.

We will characterize the selectivity of the caspase-1, -4 and -5 inhibitors for each of the wild-type enzymes. Our data shows that we can obtain compounds that are >50-fold selective for caspase-1 over caspase-4 and -5. We will add compounds at concentrations 100-fold above the DR₅₀ for the parent enzyme, and determine their extent of conjugation and inhibition for the off-target caspases. We will rank hits by their DR₅₀ for their parent enzyme and inability to inhibit the two other off-target inflammatory caspases, as well as for caspase-7 which will serve as a sentinel apoptotic caspase. In the event that we do not identify several compounds for each caspase that show >10-fold selectivity over the others with good DR₅₀ values we will take the best compounds and make small libraries (20-40 compounds each) to obtain the selectivity we desire (as described in Example 17). In multiple other cases we have found simple fragment SAR to yield compounds of higher affinity and selectivity (see Erlanson, D. A. et al., Annu. Rev. Biophys. Biomol. Struct., 33:199-223 (2004)). Upon completion of this step we will have the necessary compounds to proceed to Example 23.

### Example 23

### Determination of the Role of Caspases in Cell Extracts Stimulated with Different PAMP's

To avoid any issues with cellular transport of the compounds, we will conduct experiments in cell extracts that can recapitulate processing of pro-inflammatory proteins. Cytosolic fractions are readily prepared from THP-1 cells that have been primed with different PAMP's (see Martinon, F. et al., Mol. Cell, 10(2):417-26 (2002)). For example, LPS will be added to specifically induce the NAPL-1 inflammasome. Inflammasome assembly is activated by physical disruption after about 30 min, which leads to processing of pro-inflammatory cytokines. LPS stimulated extracts will be titrated with each of the caspase inhibitors to determine their impact on processing of pro-IL-1β, pro-IL-18, and the pro-caspases by Western blotting experiments.

We will use the general caspase substrate WEHD-AFC to measure total caspase activity. The basal caspase activity in extracts is undetectable without LPS stimulation, and goes up dramatically with LPS stimulation. We will determine which caspase or combination of caspases are responsible for the activity by adding different inhibitors either alone or in combination. We can also determine if there is an order to the processing. For example, if caspase-1 inhibitors block all caspase activity and all protein processing whereas caspase-5 inhibitors only partially block caspase activity and processing that would suggest that caspase-1 is above caspase-5 in the signaling cascade but that both are needed for pro-inflammatory processing. Thus, these tools can be useful for establishing which is responsible for the primary and secondary processing events, as well as which caspase dominates the processing. These studies and the ones below will be conducted by stimulating with different PAMP's to determine the role of each of the caspases in these processes.

There are a number of control experiments that can be performed to show these effects are specific for binding at the allosteric site of the specific caspase. First, we will add to these extracts an equivalent amount of the pro-caspase C331A allosteric site mutant. This mutant should be ˜100 fold less sensitive to the effects of the compounds, and activity and processing should be restored. On the other hand if these effects are driven by compounds binding to another protein then we should not be able to restore the system with these mutants. We will determine the IC₅₀ for several of the caspase inhibitors and see how the SAR in extracts corresponds to the SAR we observed in vitro. If we see a dramatically different order of potencies we would be suspicious that compounds are acting in extracts through a different mechanism than was seen in vitro. If we find that the compound potencies or specificities are not sufficient to pass these specificity controls, we would be inclined to further improve the compounds by additional chemical analoging. This can be readily done by making small and focused libraries around the best hits, or by screening for additional hits from the library (see Example 17). We would measure their effects in vitro (Example 13) and then test them in the extract assay.

It will be interesting to determine if the effects we observe by specific allosteric site inhibitors are the same or different from partial ablation of the caspase by siRNA knock-downs. siRNA's will be produced to each of the relevant inflammatory caspases and extracts will be prepared from LPS/ATP stimulated THP-1 cells. Levels of pro-inflammatory cytokines will be assessed by Western blotting as above. It is quite possible that we would see different effects. For example, the allosteric and active site compounds could give the same effects but with more potent IC₅₀ values than seen in vitro owed to the dominant negative effect of the CARD scaffolding domains blocking binding of non-active site occupied caspases.

### Example 24

### Determination of the Role of Caspase-4 and -5 in Pro-Inflammatory Protein Processing in Intact THP-1 Cells

We will test caspase-4 and caspase-5 selective inhibitors in THP-1 cells for their effects on pro-inflammatory protein processing in a manner similar to that proposed for the caspase-1 allosteric inhibitors in Example 18. If we observe that inhibiting either caspase-1 or caspase-5 (but not caspase-4) is sufficient to block all processing, then that would suggest caspase-1 and -5 are both needed in the processing of each other as well as the pro-inflammatory cytokines. If however, we see no effect with either caspase-4 or -5 inhibitors that would suggest neither caspase-4 or -5 can replace the function of caspase-1. Many other combinations are possible and it's uncertain at this point which will be the case. Depending on the results, it may also be informative to compare the effect of allosteric site inhibition to those obtained with siRNA knock-downs since the latter would eliminate each caspase protein (including their CARD domain scaffolding function).

Depending on the results above, we can also employ a reconstitution system in 293T cells, for which one can assemble the inflammasome by simple transfection of genes encoding two scaffolding proteins NALP-1 and ASC, plus any of the pro-caspase genes. This reconstitution system has been described (see Martinon, F. et al., Mol. Cell, 10(2):417-26 (2002)). It has been shown that LPS treatment of these transfected cells will induce transcription of pro-inflammatory cytokines which can then be processed by the transfected inflammasome components (see Hersh, D. et al., Proc Natl Acad Sci USA, 96(5):2396-401 (1999)). In this artificial overexpression system, one can test the impact of mutated inflammatory caspases for their activities in the absence of the endogenous enzymes. This has the advantage of reducing the background activity of endogenous inflammatory caspases seen in the THP-1 cells. We can also use this system to test the impact of mutations we have introduced in the allosteric circuitry of each enzyme on the ability to process the pro-inflammatory proteins in cells. For example, we could determine how k_{cat} or K_{M} mutants in each caspase affect the processing relative to their performance in vitro. Overall these studies will greatly clarify the individual roles of the caspases in inflammosome assembly and processing.

### CONCLUSIONS

The above Examples provide a new way of trapping allosteric transitions in proteins using small molecules so that these states may be more clearly studied in vitro and in cells. Our work explored this approach by trapping active and inactive states in the caspases. This will allow us to better define the internal allosteric circuitry in these enzymes that supports protein conformation and a general mechanism that applies to the inflammatory caspase family. This site may be used by a natural ligand (protein, metabolite, etc.). The site-directed nature of the disulfide trapping method allows it to be broadly applied to labeling putative allosteric sites in proteins and the caspase family represents a paradigm for this approach. New computational methods have been developed to predict allosteric sites in proteins (for example, Suel, G. et al., Nature Struct. Biol., 10(1):59-69 (2003); Lichtarge, O et al., Journal of Molecular Biology, 257:342-358 (1996); Ota, N. and D. Agard, J Mol Biol, 351(2):345-354 (2005)), and the disulfide trapping method is well suited to test them empirically. The substrate specificity of the inflammatory caspases is so close that it has been challenging to produce selective inhibitors for each of them. These studies will provide selective inhibitors for the inflammatory caspases which will be useful tools for dissecting their roles in cellular inflammation. Despite tremendous interest in the pharmaceutical industry to build drugs that target the inflammatory caspases, efforts directed toward the active sites have failed to yield compounds with good drug-like properties owing to the strict requirement for electrophilic warheads and an aspartyl functionality in the active sites. The methods disclosed herein will go far to validate the allosteric site as a viable alternative that may be more tractable than the active site. These studies will generate specific assays for triaging compounds found by HTS for those that bind the active site versus the allosteric site. Compounds identified from HTS or extended disulfide trapping can be used to seed drug discovery efforts to caspase-1. Lastly, the development of the disulfide-trapping technology for producing cell active compounds will have a dramatic impact on being able to generate site-selective modulators of proteins in cells or cell extracts.

It is to be understood that the above description is intended to be illustrative and not restrictive. Many embodiments will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patent applications, patents, PCT publications, and Accession Nos. are incorporated herein by reference for all purposes.

What is claimed is:

1. A method of generating a protein binding domain that specifically binds to a protein in a specific conformational state, the method comprising the steps of:
a) contacting a protein or a fragment thereof with a modifying agent that fixes the conformational state of the protein; and
b) generating a protein binding domain that binds to the protein bound to the modifying agent,
wherein the protein binding domain is specific for the conformational state of the protein.

2. The method of claim 1, wherein the protein binding domain is selected from the group consisting of: an antibody, protein A, protein G, anchorin repeat domains, Fibronectin III domains, DNA, and RNA.

3. The method of claim 1, wherein the protein binding domain is an antibody.

4. The method of claim 3, wherein the antibody is monoclonal.

5. The method of claim 3, wherein the antibody is polyclonal.

6. The method of claim 1, wherein the protein is selected from the group consisting of: an inflammatory protein, a metabolic enzyme, a programmed cell death protein, a G-protein coupled receptor, an antibody, a blood coagulation factor, a cellular receptor, a coagulation factor, a protease, an extracellular protein or enzyme, a transcription factor, a cytoskeleton protein, a hormone receptor, a complement fixation protein, kinases and phosphatases.

7. The method of claim 6, wherein the programmed cell death protein is selected from the group consisting of caspase 1, 3, 4, 5, and 7.

8. The method of claim 6, wherein the G-protein coupled receptor is a C5a receptor.

9. The method of claim 1, wherein the modifying agent is an agent that reacts a group on the protein selected from the group consisting of thiol, amino, and carboxyl groups.

10. The method of claim 1, wherein the binding of the modifying agent to the protein is reversible.

11. The method of claim 1, wherein the binding of the modifying agent to the protein is irreversible.

12. The method of claim 1, wherein the conformational state of the protein is active.

13. The method of claim 1, wherein the conformational state of the protein is inactive.

14. A method of decreasing the activity of a protein comprising the step of contacting the protein with the protein binding domain of claim 1.

15. A method of increasing the activity of a protein comprising the step of contacting the protein with the protein binding domain of claim 1.

16. A method of generating an antibody that specifically binds to a protein in a specific conformational state, the method comprising the steps of:
a) contacting a protein or a fragment thereof with a modifying agent that fixes the conformational state of the protein; and
b) generating antibodies to the protein bound to the modifying agent,
wherein the antibodies are specific for the conformational state of the protein.

17. A method for diagnosing a disease in a subject comprising contacting a sample from the subject with the protein binding domain of claim 1, wherein the protein binding domain binds to a form of the protein present in the disease and is indicative of presence of the disease in the subject.

18. The method of claim 17, wherein the disease is selected from the group consisting of: cancer, autoimmune disease, Parkinson's disease, stroke, myocardial infarction, chronic inflammation, prion infection, neurological disease, renal disease, and infectious disease.

19. The method of claim 17, wherein the protein binding domain is selected from the group consisting of: an antibody, protein A, protein G, anchorin repeat domains, Fibronectin III domains, DNA, and RNA.

20. The method of claim 17, wherein the protein binding domain is an antibody.

21. A kit for diagnosing a disease comprising the protein binding domain of claim 17.

22. A method of treating or preventing a disease, the method comprising the step of administering a therapeutically effective amount of the protein binding domain of claim 1.

23. The method of claim 22, wherein the disease is selected from the group consisting of: cancer, autoimmune disease, Parkinson's disease, stroke, myocardial infarction, chronic inflammation, prion infection, neurological disease, renal disease, and infectious disease.

24. A method of purifying a protein in a specific conformational state, the method comprising the steps of:
a) contacting a population of proteins with a plurality of conformational states with the protein binding domain of claim 1;
b) isolating the complex of the protein binding domain bound to the protein; and
c) eluting the protein from the protein binding domain,
wherein at least 50% of the resulting protein is in the specific conformational state.

25. The method of claim 24, wherein the protein is a vaccine, a therapeutic protein, or an antibody.

26. A method for screening for compounds that induce a specific conformational state of a protein, the method comprising the steps of:
a) contacting a test compound with the protein;
b) contacting the protein in the presence or absence of the test compound with the protein binding domain of claim 1; and
c) detecting the binding of the antibody to the protein,
wherein increased binding of the protein binding domain to the protein in the presence of the compound as compared to when the compound is absent indicates the adoption of the specific conformational state by the protein in the presence of the test compound.
